SEQUENCE LISTING (17 sequences)

SEQ ID NO: 1 - protein, 120 aa, Homo sapiens
QVQLQQSGPELVKPGASVKLSCKASGYTFTSYDINWVKQRPGQGLEWIGWIYPRDGSIKFNEKFKGKATLTVDTSSSTAYMELHSLTSEDSAAYFCARRGRLLLYGFAYWGQGTLVTVSA

SEQ ID NO: 2 - protein, 114 aa, Homo sapiens
DVVMTQLPLSLPVILGDQASISCRSSQSLIHSNGNTYLHWYLQKPGQSPKLLIYKVSNRFSGVPDRFSGSGSGTDFTLKISRVEAEDLGVYFCSQSTHVPFTFGSGTKLEIKRA

SEQ ID NO: 3 - protein, 116 aa, Homo sapiens
QVQLQQSGAELVRPGASVTLSCKASGYTFTDYEMHWVKQTPVHGLEWIGTIDPETAGTAYNQKFKGKAILTAGKSSSTAYMELRSLTSEDSAVYYCTGVTTWFAYWGQGTLVTVSA

SEQ ID NO: 4 - protein, 114 aa, Homo sapiens
DVVMTQTPLSLPVSLGDQASISCRSSQSLVHSNGNTYLHWYLQKPGQSPNLLIYKVSNRFSGVPDRFSGSGSGTDFTLKISRVEAEDLGVYFCSQGTHVPYTFGGGTKLEIKRA

SEQ ID NO: 5 - protein, 11 aa, Homo sapiens
RGRLLLYGFAY

SEQ ID NO: 6 - protein, 9 aa, Homo sapiens
SQSTHVPFT

SEQ ID NO: 7 - protein, 7 aa, Homo sapiens
VTTWFAY

SEQ ID NO: 8 - protein, 9 aa, Homo sapiens
SQGTHVPYT

SEQ ID NO: 9 - protein, 5 aa, Homo sapiens
SYDIN

SEQ ID NO: 10 - protein, 16 aa, Homo sapiens
RSSQSLIHSNGNTYLH

SEQ ID NO: 11 - protein, 5 aa, Homo sapiens
DYEMH

SEQ ID NO: 12 - protein, 16 aa, Homo sapiens
RSSQSLNHSNGNTYLH

SEQ ID NO: 13 - protein, 17 aa, Homo sapiens
WIYPRDGSIKFNEKFKG

SEQ ID NO: 14 - protein, 7 aa, Homo sapiens
KVSNRFS

SEQ ID NO: 15 - protein, 17 aa, Homo sapiens
TIDPETAGTAYNQKFKG

SEQ ID NO: 16 - protein, 7 aa, Homo sapiens
KVSNRFS

SEQ ID NO: 17 - protein, 144 aa, Homo sapiens
PTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREH

(19) United States
(12) Patent Application Publication
(10) Pub. No.: US 2010/0297115 A1
(43) Pub. Date: Nov. 25, 2010

(54) USE OF TRKB ANTIBODIES FOR THE TREATMENT OF RESPIRATORY DISORDERS

(75) Inventor: Cecile Blaustein, Cambridge, MA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: Novartis AG

(21) Appl. No.: 12/739,438

(22) PCT Filed: Oct. 23, 2008
(86) PCT No.: PCT/EP08/64391

§ 371 Date: Apr. 23, 2010

Related U.S. Application Data
(60) Provisional application No. 60/981,851, filed on Oct. 23, 2007.

(51) Int. Cl. A61K 39/395

(52) U.S. Cl. 424/133.1; 424/130.1

(57) ABSTRACT

The invention discloses TrkB antibodies for the development of new therapeutics to treat, prevent or ameliorate respiratory disorders. The invention also relates to methods to treat, prevent or ameliorate said conditions and pharmaceutical compositions therefor, as well as to a method to identify compounds with therapeutic usefulness to treat conditions associated with respiratory disorders.

### BACKGROUND OF THE INVENTION

Tyrosine kinase receptor B (TrkB) belongs to a family of single transmembrane receptor tyrosine kinases that includes TrkA and TrkC. These tyrosine kinase receptor s (trks) mediate the activity of neurotrophins. Neurotrophins are required for neuronal survival and development and regulate synaptic transmission via modulation of neuronal architecture and synaptic plasticity. Neurotrophins include, but are not limited to, nerve growth factor (NGF), brain derived neurotrophic factor (BDNF), neurotrophin-3 (NT-3), and neurotrophin-4/5 (NT-4/5). (Lo, K Y et al., (2005) J. Biol. Chem., 280:41744-52).

TrkB is a high affinity receptor of BDNF (Minichiello, et al., Neuron 21:335-45 (1998)), and is highly expressed in the brain, particularly in the neocortex, hippocampus, striatum, and brainstem; an isoform can also be found in skeletal muscle. Neurotrophin binding to trk activates the receptor, which dimerizes and auto-phosphorylates specific tyrosine residues on the intracellular domain of the receptor (Jing, et al., (1992) Neuron 9:1067-1079; Barbacid, (1994) J. Neurobiol. 25:1386-1403; Bothwell, (1995) Ann. Rev. Neurosci. 18:223-253; Segal and Greenberg, (1996) Ann. Rev. Neurosci. 19:463-489; Kaplan and Miller, (2000) Curr. Opinion Neurobiol. 10:381-391). These phospho-tyrosine residues serve as docking sites for elements of intracellular signaling cascades that lead to the suppression of neuron death, the promotion of neurite outgrowth, and other effects of the neurotrophins. For example, Shc, FRS-2, SH2B, rAPS, and PLCγ interact with TrkB via phosphorylated tyrosine residues. Association of these adaptor molecules with activated TrkB results in the initiation of signaling pathways, including the mitogen-activated protein kinase, phosphatidylinositol 3-kinase, and PLCγ pathways, thereby mediating the actions of neurotrophins (Lo, K Y et al., (2005) J. Biol. Chem., 280:41744-52).

First identified by Dr. Andreas Rett in 1966, Rett Syndrome (RTT) is a devastating CNS disorder that originates from late-neurodevelopmental defects. It almost exclusively affects young girls of all ethnicities at a rate of 1/10,000-15,000 live births. Some individuals with RTT die at a young age; many, however, can live into adulthood and are profoundly disabled. Up to 25% of patients die of cardiac/respiratory failures. There is so far no effective treatment for the disease.

Following a period of apparent normal development, affected girls develop RTT symptoms at the age of 6-18 months, which progressively worsen over the next few years. Symptoms include normal head circumference at birth followed by deceleration of head growth; loss of acquired speech, communication dysfunction, cognitive impairment; purposeful hand skills replaced by stereotypical hand movements (tortuous hand wringing, hand washing, clapping, patting, etc.); impaired or deteriorating locomotion (gait ataxia, stiffness, etc.); breathing difficulties while awake; impaired sleeping pattern from early infancy; abnormal muscle tone accompanied by muscle wasting and dystonia; peripheral vasomotor disturbances; progressive scoliosis or kyphosis; and growth retardation.

The disorder is also characterized by central autonomic dysfunctions, and Rett patients exhibit some or all of the following symptoms: multiple respiratory dysrhythmias consisting of periods of breath holding, shallow breathing, hyperventilation, prolonged apneas; cardiac arrhythmias with reduction in baseline cardiac vagal tone; and cardiac sensitivity to baroreflex. These symptoms are life-threatening and render Rett patients at risk of sudden death. They indicate brainstem immaturity and a lack of integrative inhibition within the cardiorespiratory network and from the hypothalamus or limbic cortex during wakefulness. Furthermore, alteration in brain stem neurotrophin signaling (NGF and BDNF) is reported in Rett patients, as is reduction in monoamine (serotonin, norepinephrine) and neuropeptide (Substance P) levels.

RTT is a monogenic disease, caused in the vast majority of cases by mutations in the X chromosome-linked gene mecp2, which encodes the transcriptional repressor MeCP2 (methyl-CpG cytosine binding protein 2). MeCP2 binds preferentially to methylated DNA.

The Neurotrophin factor BDNF is a known direct target of MeCP2, and is an important trophic factor for norepinephrine and serotonin neurons. Surprisingly, Mecp2-KO mice are deficient in brain BDNF, and a genetic overexpression of brain BDNF can increase their lifespan and rescue some of their locomotor defects. There exists a present need to seek BDNF therapy strategies, including for conditions such as Rett Syndrome and other respiratory disorders; among such strategies are targeting such binding partners of BDNF as the tyrosine kinase receptor TrkB.

### SUMMARY OF THE INVENTION

The present invention provides methods of treating, diagnosing, preventing, and/or ameliorating respiratory disorders (e.g., Rett Syndrome (RTT)), comprising administering isolated antibody agonists of Tyrosine Kinase Receptor B (TrkB) (also referred to herein as “TrkB agonizing antibodies,” “TrkB binding molecules,” and the like). In some embodiments, the antibody is a humanized antibody. In other embodiments, the antibody is a single chain antibody. In some embodiments, the antibody does not bind to Tyrosine Kinase Receptor A or Tyrosine Kinase Receptor C. In some embodiments, the antibody is capable of binding the human version of TrkB, and not to the TrkB of other species. In some embodiments, the antibody is capable of binding the human version of TrkB, and to the TrkB of other species as well (i.e., is capable of cross-reactivity)(including, e.g., to mouse, rat, and/or non-human primate (e.g., a cynomolgus monkey, or a rhesus monkey)).

In some embodiments, the antibody binds to the Ligand Binding Domain (LBD) of TrkB. In some embodiments, the antibody competes with the binding of Brain Derived Neurotrophic Factor (BDNF) to TrkB. In some embodiments, the antibody competes for binding to TrkB with a competitor antibody comprising a heavy chain variable region comprising SEQ ID NO:3 and a light chain variable region comprising SEQ ID NO:4. In some embodiments, the antibody comprises a heavy chain variable region comprising SEQ ID NO:7 and a light chain variable region comprising SEQ ID NO:8. In some embodiments, the antibody comprises a heavy chain variable region comprising SEQ ID NO:11 and a light chain variable region comprising SEQ ID NO:12. In some embodiments, the antibody comprises a heavy chain variable region comprising SEQ ID NO:15 and a light chain variable region comprising SEQ ID NO:16. In some embodiments, the antibody comprises a combination of heavy chain variable regions comprising SEQ ID NO:7, SEQ ID NO:11, and/or SEQ ID NO:15; and light chain variable regions comprising SEQ ID NO:8, SEQ ID NO:12, and/or SEQ ID NO:16. In some embodiments, the antibody comprises a heavy chain variable region comprising SEQ ID NO:3 and a light chain variable region comprising SEQ ID NO:4.

In some embodiments, the antibody of the present methods acts as a BDNF mimetic, and is capable of, e.g., recapitulating the trophic activities of said ligand (and therefore, is capable of exerting neuroprotective and neurotrophic effects).

In some embodiments, the antibody does not bind to the Ligand Binding Domain (LBD) of TrkB. In some embodiments, the antibody does not compete with the binding of Brain Derived Neurotrophic Factor (BDNF) to TrkB. In some embodiments, the antibody competes for binding to TrkB with a competitor antibody comprising a heavy chain variable region comprising SEQ ID NO:1 and a light chain variable region comprising SEQ ID NO:2. In some embodiments, the antibody comprises a heavy chain variable region comprising SEQ ID NO:5 and a light chain variable region comprising SEQ ID NO:6. In some embodiments, the antibody comprises a heavy chain variable region comprising SEQ ID NO:9 and a light chain variable region comprising SEQ ID NO:10. In some embodiments, the antibody comprises a heavy chain variable region comprising SEQ ID NO:13 and a light chain variable region comprising SEQ ID NO:14. In some embodiments, the antibody comprises a combination of heavy chain variable regions comprising SEQ ID NO:5, SEQ ID NO:9, and/or SEQ ID NO:13; and light chain variable regions comprising SEQ ID NO:6, SEQ ID NO:10, and/or SEQ ID NO:14. In some embodiments, the antibody comprises a heavy chain variable region comprising SEQ ID NO:1 and a light chain variable region comprising SEQ ID NO:2.

TrkB agonizing antibodies interact with TrkB and are thereby capable of modulating TrkB functions. TrkB agonizing binding molecules can be used to facilitate TrkB pathway signaling; therefore, TrkB agonizing binding molecules can be used to e.g., diagnose, ameliorate the symptoms of, protect against, and treat respiratory disorders associated with aberrantly low levels of TrkB pathway signaling (e.g., due to a mutated version of TrkB or one of its protein interactors in an afflicted subject). Non-limiting examples of disorders associated with aberrant downregulation of TrkB signaling, e.g., due to a mutated version of TrkB or one of its protein interactors, are (i) Rett Syndrome (RTT), which is characterized by mutations in the gene encoding MeCP2 (which binds directly to BDNF); and (ii) severe obesity and developmental delay, due to a TrkB loss-of-function mutation. (Giles, S., et al. (2004) Nature Neuroscience 7:1187-9).

The present invention also provides methods of treating, diagnosing, preventing, and/or ameliorating respiratory disorders (e.g., Rett Syndrome (RTT)), comprising administering pharmaceutical compositions comprising a therapeutically or prophylactically effective amount of the TrkB agonizing antibodies; and a pharmaceutical carrier. In some embodiments, the pharmaceutical composition further comprises a separate and independent agent that is capable of treating, diagnosing, preventing, and/or ameliorating symptoms of respiratory distress (e.g., breathing difficulties), such as small molecule activators of the norepinephrine and/or serotonin pathways (examples are the tricyclic antidepressant desipramine (DMI), the serotonin 1A receptor partial agonist, buspirone, and potentially the more selective antidepressants Fluoxetine and Reboxetine), the activator of glutamatergic AMPA receptors: AMPAkine CX546, prostaglandin, progesterone, or potentiators of TrkB activity (e.g., protein tyrosine phosphatase inhibitors).

In some embodiments, a therapeutically and/or prophylactically effective amount of a second agent effective in treating, diagnosing, preventing, and/or ameliorating respiratory disorders (e.g., Rett Syndrome (RTT)), or symptoms of respiratory distress, is administered to the individual in combination with the antibody agonist of TrkB (or pharmaceutical composition containing the same). In some embodiments, the second agent and the antibody agonist of TrkB (or pharmaceutical composition containing the same) are administered as a mixture. In some embodiments, the second agent is selected from the group consisting of small molecule activators of the norepinephrine and/or serotonin pathways (examples are the tricyclic antidepressant desipramine (DMI), the serotonin 1A receptor partial agonist, buspirone, and potentially the more selective antidepressants Fluoxetine and Reboxetine), the activator of glutamatergic AMPA receptors: AMPAkine CX546, prostaglandin, progesterone, or potentiators of TrkB activity (e.g., protein tyrosine phosphatase inhibitors).

The present invention also provides methods of treating, diagnosing, preventing, and/or ameliorating symptoms of respiratory distress, such as those commonly found with respiratory disorders, comprising administering TrkB agonizing antibodies (or pharmaceutical compositions comprising the same). Said symptoms include but are not limited to breathing difficulties (e.g., stridor or wheezing, breath holding, shallow breathing, hyperventilation, prolonged apneas), poor or decreased oxygenation of the blood (e.g., cyanosis)(e.g., due to impaired absorption of oxygen, inadequate perfusion of the lungs with blood, etc.), reduced norepinephrine (NE) content, decrease of tyrosine hydroxylase (TH)-expressing neurons in the medulla, and chest pain. In some embodiments, said methods comprise administering a therapeutically or prophylactically effective amount of an antibody agonist of Tyrosine Kinase Receptor B (TrkB) to the individual. In some embodiments, said methods comprise administering a pharmaceutical composition comprising a therapeutically or prophylactically effective amount of TrkB agonizing antibody and a pharmaceutical carrier to the individual. In some embodiments, the individual has one or more respiratory disorders and/or is experiencing one or more symptoms of respiratory distress. In some embodiments, the individual is predisposed to symptoms of respiratory distress. In some embodiments, the individual has Rett Syndrome.

The present invention provides methods of suppressing neural cell death, comprising administering TrkB agonizing antibodies (or pharmaceutical compositions comprising the same). In some embodiments, the antibody is a humanized antibody. In other embodiments, the antibody is a single chain antibody. In some embodiments, the antibody does not bind to Tyrosine Kinase Receptor A or Tyrosine Kinase Receptor C. In some embodiments, the antibody does not bind to neurotrophin receptor p75NR. In some embodiments, the antibody is capable of binding the human version of TrkB, and not to the TrkB of other species. In some embodiments, the antibody is capable of binding the human version of TrkB, and to the TrkB of other species as well (i.e., is capable of cross-reactivity)(including, e.g., to mouse, rat, and/or non-human primate (e.g., a cynomolgus monkey, or a rhesus monkey)).

In some embodiments, the antibody is a humanized antibody.

In various embodiments, the invention provides methods of treating, diagnosing, preventing, and/or ameliorating respiratory disorders (e.g., Rett Syndrome (RTT)), or symptoms of respiratory distress, or methods of suppressing neural cell death, with TrkB agonizing antibodies that modulate (e.g., promote) one or more biological functions of TrkB. For example, a TrkB agonist antibody can modulate dimerization of TrkB, and subsequent auto-phosphorylation of specific tyrosine residues on the TrkB intracellular domain. By way of further example, a TrkB agonist antibody can initiate TrkB-related intracellular signaling cascades (e.g., the mitogen-activated protein kinase, phosphatidylinositol 3-kinase, and PLCγ pathways) that lead to the suppression of neuron death, the promotion of neurite outgrowth, and other effects of the neurotrophins.

TrkB agonizing antibodies include, for example, antibodies that bind to TrkB (e.g., within a particular domain or epitope of TrkB, such to Ligand Binding Domain of TrkB, or outside of the Ligand Binding Domain), and polypeptides that include antigen binding portions of such antibodies. TrkB agonizing antibodies also include molecules in which the binding portion is not derived from an antibody, e.g., TrkB agonizing antibodies derived from polypeptides that have an immunoglobulin-like fold, and in which the antigen binding portion is engineered to bind TrkB through randomization, selection, and affinity maturation.

In various embodiments, the invention provides methods of treating, diagnosing, preventing, and/or ameliorating respiratory disorders (e.g., Rett Syndrome (RTT)), or symptoms of respiratory distress, or methods of suppressing neural cell death, with TrkB agonist antibodies that bind to an epitope within human TrkB and which are cross reactive with the TrkB protein (or portion thereof) of a non-human primate (e.g., a cynomolgus monkey, or a rhesus monkey). In various embodiments, said TrkB agonist antibody is cross reactive with TrkB of a rodent species (e.g., murine TrkB, rat TrkB). In various embodiments, said TrkB agonist antibody is cross reactive with human TrkA or TrkC.

In other embodiments, the invention provides methods of treating, diagnosing, preventing, and/or ameliorating respiratory disorders (e.g., Rett Syndrome (RTT)), or symptoms of respiratory distress, or methods of suppressing neural cell death, with TrkB antibodies that binds to an epitope within human TrkB but which are not cross reactive with the TrkB protein (or portion thereof) of a non-human primate (e.g., a cynomolgus monkey, or a rhesus monkey). In various embodiments, said TrkB agonist antibody is not cross reactive with TrkB of a rodent species (e.g., murine TrkB, rat TrkB). In various embodiments, said TrkB agonist antibody does not cross react with human TrkA or TrkC, or with neurotrophin receptor p75NR.

In various embodiments, the antigen binding portion of a TrkB agonizing antibody of the present methods binds to a linear epitope. In various embodiments, said antigen binding portion binds to a non-linear epitope.

In various embodiments, the antigen binding portion of a TrkB agonizing antibody of the present methods binds to TrkB with a dissociation constant (K_{D}) equal to or less than 1 nM, 0.5 nM, 0.25 nM, or 0.1 nM.

In some embodiments, the antibody is capable of binding the human version of TrkB, and not to the TrkB of other species. In some embodiments, the antibody is capable of binding the human version of TrkB, and to the TrkB of other species as well (i.e., is capable of cross-reactivity)(including, e.g., to mouse, rat, and/or non-human primate (e.g., a cynomolgus monkey, or a rhesus monkey)).

In various embodiments, the antigen binding portion of a TrkB agonizing antibody of the present methods binds to TrkB of a non-human primate (e.g., cynomolgus monkey or chimpanzee) with a K_{D} equal to or less than 0.3 nM.

In various embodiments, said antigen binding portion binds to mouse TrkB with a K_{D} equal to or less than 0.5 nM.

In one embodiment, the TrkB agonizing antibody of the present methods is a human antibody. In another embodiment, said TrkB agonist antibody is a non-human antibody. In another embodiment, said TrkB agonist antibody is a chimeric (e.g., humanized, humaneered) antibody.

In one embodiment, the antigen binding portion of a TrkB agonizing antibody of the present methods is an antigen binding portion of a human antibody. Said antigen binding portion can be an antigen binding portion of a monoclonal antibody or a polyclonal antibody.

The TrkB agonizing antibody of the present methods includes, for example, a Fab fragment, a Fab′ fragment, a F(ab′)₂, or an Fv fragment of the antibody.

In some embodiments, the TrkB agonist antibody of the present methods is pegylated. In some embodiments, the TrkB agonist antibody is a pegylated Fab fragment.

In one embodiment, the TrkB agonist antibody of the present methods includes a single chain Fv.

In one embodiment, the TrkB agonist antibody of the present methods includes a diabody (e.g., a single chain diabody, or a diabody having two polypeptide chains).

In some embodiments, the antigen binding portion of the TrkB agonist antibody of the present methods is derived from an antibody of one of the following isotypes: IgG1, IgG2, IgG3 or IgG4. In some embodiments, the antigen binding portion of said antibody is derived from an antibody of an IgA or IgE isotype.

The TrkB agonist antibody of the present methods can exhibit one or more of a number of biological activities. In various embodiments, the TrkB agonist antibody inhibits TrkB binding to BDNF, to neurotrophin 4 (NT-4), and/or to neurotrophin 5 (NT-5). For example, the TrkB agonist antibody inhibits TrkB binding to BDNF, NT-4, and/or NT-5 by at least 5%, 10%, 15%, 25%, or 50%, relative to a control (e.g., relative to binding in the absence of the TrkB agonist antibody). In other embodiments, the TrkB agonist antibody does not inhibit, and in no way competes with, TrkB binding to BDNF, NT-4, and/or NT-5.

In one embodiment, a TrkB agonist antibody of the present methods competes with BDNF for binding to TrkB, thereby modulating the biological activity and consequences of TrkB pathway signaling. By way of non-limiting example, a TrkB agonist antibody of the present methods can activate, enhance, or perpetuate TrkB pathway activation and signaling (e.g., by competing with BDNF for binding to TrkB). In some embodiments, said TrkB agonist antibody binds to the TrkB Ligand Binding Domain and thereby competes with BDNF for binding to TrkB.

In some embodiments, the antibody of the present methods acts as a BDNF mimetic, and is capable of e.g., recapitulating the trophic activities of said ligand (and therefore, is capable of exerting neuroprotective and neurotrophic effects).

In other embodiments, the TrkB agonist antibody of the present methods does not bind to the TrkB Ligand Binding Domain, and does not compete with BDNF for binding with TrkB, but is capable nevertheless of modulating the TrkB signaling pathway (e.g., activating, enhancing, or perpetuating TrkB pathway activation and signaling).

In various embodiments, the invention provides methods of treating, diagnosing, preventing, and/or ameliorating respiratory disorders (e.g., Rett Syndrome (RTT)), or symptoms of respiratory distress, or methods of suppressing neural cell death, with TrkB agonizing antibodies which modulate downstream biological activities normally modulated in a direct or indirect fashion by TrkB. Non-limiting examples of said activities include modulating dimerization of TrkB, and subsequently auto-phosphorylating tyrosine residues on the TrkB intracellular domain; initiating TrkB-related intracellular signaling cascades such as the mitogen-activated protein kinase, phosphatidylinositol 3-kinase, and PLCγ pathways; and suppressing of neuron death, the promotion of neurite outgrowth, and other effects of the neurotrophins. For example, a TrkB agonist antibody of the present methods suppresses neuron death by at least a 5%, 10%, 15%, 25%, or 50%, greater margin relative to a control (e.g., relative to activity in the absence of the TrkB agonist antibody). By way of further example, said TrkB agonist antibody stabilizes TrkB protein levels by at least a 5%, 10%, 15%, 25%, or 50%, greater margin relative to a control (e.g., relative to activity in the absence of the TrkB agonist antibody).

In various embodiments, the invention provides methods of treating, diagnosing, preventing, and/or ameliorating respiratory disorders (e.g., Rett Syndrome (RTT)), or symptoms of respiratory distress, or methods of suppressing neural cell death, with non-antibody TrkB agonizing molecules. A non-antibody TrkB agonist molecule includes a TrkB binding domain that has an amino acid sequence derived from an immunoglobulin-like (Ig-like) fold of a non-antibody polypeptide, such as one of the following: tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class I WIC, T-cell antigen receptor, CD1, C2 and I-set domains of VCAM-1, I-set immunoglobulin domain of myosin-binding protein C, I-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, or thaumatin. In general, the amino acid sequence of the TrkB binding domain is altered, relative to the amino acid sequence of the immunoglobulin-like fold, such that the TrkB binding domain specifically binds to the TrkB (i.e., wherein the immunoglobulin-like fold does not specifically bind to the TrkB).

In various embodiments, the amino acid sequence of the TrkB binding domain is at least 60% identical (e.g., at least 65%, 75%, 80%, 85%, or 90% identical) to an amino acid sequence of an immunoglobulin-like fold of a fibronectin, a cytokine receptor, or a cadherin.

In various embodiments, the amino acid sequence of the TrkB binding domain is at least 60%, 65%, 75%, 80%, 85%, or 90% identical to an amino acid sequence of an immunoglobulin-like fold of one of the following: tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class I MHC, T-cell antigen receptor, CD1, C2 and I-set domains of VCAM-1, I-set immunoglobulin domain of myosin-binding protein C, I-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, or thaumatin.

In various embodiments, the TrkB binding domain binds to TrkB with a K_{D} equal to or less than 1 nM (e.g., 0.5 nM, 01 nM).

In some embodiments, the Ig-like fold is an Ig-like fold of a fibronectin, e.g., an Ig-like fold of fibronectin type III (e.g., an Ig-like fold of module 10 of fibronectin III).

The invention also features methods of using pharmaceutical compositions that include a TrkB agonist antibody described herein. The composition includes, for example, a TrkB agonist antibody and a pharmaceutically acceptable carrier.

In one aspect, the invention features methods of suppressing neural cell death or the promoting of neurite outgrowth by administering a therapeutically and/or prophylactically effective amount of an antibody agonist of TrkB (or pharmaceutical composition containing the same). These methods includes contacting tissues or biological samples (e.g., TrkB-expressing neuronal cells) with a therapeutically and/or prophylactically effective amount of an antibody agonist of TrkB (or pharmaceutical composition containing the same), thereby activating and/or stabilizing the TrkB signaling pathway, and protecting against the effects of neurotrophins such as BDNF. The TrkB agonist antibody (or pharmaceutical composition containing the same) can be administered in an amount effective to suppress neural cell death or promote of neurite outgrowth.

In some embodiments, the methods feature intra-peritoneal injection administration of the antibody agonist of TrkB (or pharmaceutical composition containing the same).

The details of one or more embodiments of the invention are set forth in the accompanying drawings and the description below. Other features, objects, and advantages of the invention will be apparent from the description and drawing, and from the claims.

### BRIEF DESCRIPTION OF THE DRAWINGS

FIG. 1 is a graphical depiction of the drop in body weight and food intake seen in Mecp2 knockout mice to which TrkB agonist antibodies were administered. In FIG. 1A, the top line (with white square icons as data points) represents the Mecp2 wild type mice with saline administered. The second to top line (with black square icons as data points) represents the Mecp2 wild type mice with TrkB agonist antibodies administered. The second to bottom line (with white circle icons as data points) represents the Mecp2 knockout mice with saline administered. The bottom line (with black circle icons as data points) represents the Mecp2 knockout mice with TrkB agonist antibodies administered. In FIG. 1B, the white bars represent the Mecp2 wild type mice with saline administered. The black bars represent the Mecp2 wild type mice with TrkB agonist antibodies administered. The light grey bars represent the Mecp2 knockout mice with saline administered. The dark grey bars represent the Mecp2 knockout mice with TrkB agonist antibodies administered. FIG. 1B on the Left represent the measurements taken at 6 weeks of age and on the Right, the measurements taken at 8 weeks of age

FIGS. 2A and 2B are respectively a graphical depiction of the improvement in grip strength and body fat composition in Mecp2 knockout mice to which TrkB agonist antibodies were administered. In FIG. 2, the square icons as data points represent the wild type (WT) mice treated with saline (SAL) or the TrkB agonist antibody C20; the circle icons as data points represent the knockout (KO) mice treated with saline (SAL) or the TrkB agonist antibody, C20. FIG. 2A on the left represent the hind limb measurement and on the right the forelimb measurement. FIG. 2B on the left represents the body fat content, and on the right, the lean mass content.

FIG. 3 is a graphical depiction of the increased lifespan seen in Mecp2 knockout mice to which TrkB agonist antibodies were administered. In FIG. 3A, the knockout mice are described as KO and the wild type as WT. SAL means saline was administered, and C20 means TrkB agonist antibody was administered. In FIG. 3B, The knockout mice are described as KO, and the TrkB agonist antibody is described as C20. As shown in FIG. 3, the TrkB agonist mAb-treated knockout mice (KO-C20) are able to survive to at least twice the age of the saline treated KO mice.

### DETAILED DESCRIPTION OF THE INVENTION

The present invention provides methods of treating, diagnosing, preventing, and/or ameliorating respiratory disorders (e.g., Rett Syndrome (RTT)) with isolated antibody agonists of Tyrosine Kinase Receptor B (TrkB) (also referred to herein as “TrkB agonizing antibodies,” and the like).

Said methods can employ any TrkB agonist antibodies, and those specifically listed are considered non-limiting embodiments.

The present invention provides molecules that bind to TrkB (“TrkB agonizing antibodies”), particularly human antibodies and portions thereof that bind to human TrkB and modulate its functions. Epitopes of TrkB and agents that bind these epitopes are also provided herein.

The full length sequence of human TrkB is found under Genbank® Accession Number AAB33109 (GI:913718), and has 822 residues, It is encoded by an mRNA sequence with Genbank® Accession Number S76473 (GI: 913717). TrkB is a neurotrophin receptor with wide distribution in the brain. It is a multidomain transmembrane protein that consists of an extracellular ligand binding domain (LBD), a transmembrane region, and an intracellular tyrosine kinase domain. TrkB is a high affinity receptor of BDNF, and is capable of binding neurotrophin 4 (NT-4) as well.

In some embodiments, the antibody is a humanized antibody. In other embodiments, the antibody is a single chain antibody. In some embodiments, the antibody does not bind to Tyrosine Kinase Receptor A or Tyrosine Kinase Receptor C. In some embodiments, the antibody is capable of binding the human version of TrkB, and not to the TrkB of other species. In some embodiments, the antibody is capable of binding the human version of TrkB, and to the TrkB of other species as well (i.e., is capable of cross-reactivity)(including, e.g., to mouse, rat, and/or non-human primate (e.g., a cynomolgus monkey, or a rhesus monkey)).

In some embodiments, the antibody binds to the Ligand Binding Domain (LBD) of TrkB. In some embodiments, the antibody competes with the binding of Brain Derived Neurotrophic Factor (BDNF) to TrkB. In some embodiments, the antibody competes for binding to TrkB with a competitor antibody comprising a heavy chain variable region comprising SEQ ID NO:3 and a light chain variable region comprising SEQ ID NO:4. In some embodiments, the antibody comprises a heavy chain variable region comprising SEQ ID NO:7 and a light chain variable region comprising SEQ ID NO:8. In some embodiments, the antibody comprises a heavy chain variable region comprising SEQ ID NO:11 and a light chain variable region comprising SEQ ID NO:12. In some embodiments, the antibody comprises a heavy chain variable region comprising SEQ ID NO:15 and a light chain variable region comprising SEQ ID NO:16. In some embodiments, the antibody comprises a combination of heavy chain variable regions comprising SEQ ID NO:7, SEQ ID NO:11, and/or SEQ ID NO:15; and light chain variable regions comprising SEQ ID NO:8, SEQ ID NO:12, and/or SEQ ID NO:16. In some embodiments, the antibody comprises a heavy chain variable region comprising SEQ ID NO:3 and a light chain variable region comprising SEQ ID NO:4.

In some embodiments, the antibody of the present methods acts as a BDNF mimetic, and is capable of, e.g., recapitulating the trophic activities of said ligand (and therefore, is capable of exerting neuroprotective and neurotrophic effects).

In some embodiments, TrkB agonist antibodies of the invention bind to the TrkB Ligand Binding Site and/or compete with BDNF for binding to TrkB. An exemplary antibody that binds to the ligand binding site of TrkB is Antibody A10F18.2 (also referred to herein as “A10F18” or “A10”). The heavy chain variable region of antibody A10 is exemplified in SEQ ID NO:3 and the light chain variable region of antibody A10 is exemplified in SEQ ID NO:4.

Accordingly, the invention provides agonist antibodies that compete for binding to TrkB with an antibody comprising a heavy chain variable region comprising SEQ ID NO:3 and a light chain variable region comprising SEQ ID NO:4. In some embodiments, the antibodies of the invention comprise at least one of the complementarity determining regions (CDRs) of SEQ ID NO:3 and/or 4. Without intending to limit the scope of the invention, it is believed that CDR3 plays a significant role in the binding of antibody A10. Accordingly, in some embodiments, an antibody of the present invention comprises SEQ ID NOs: 7 and/or 8. However, CDR1 and/or CDR2 also play a role in binding. Accordingly, in some embodiments, an antibody of the present invention comprises SEQ ID NOs: 11 and/or 12 or 15 and/or 16.

In some embodiments, the antibody does not bind to the Ligand Binding Domain (LBD) of TrkB. In some embodiments, the antibody does not compete with the binding of Brain Derived Neurotrophic Factor (BDNF) to TrkB. In some embodiments, the antibody competes for binding to TrkB with a competitor antibody comprising a heavy chain variable region comprising SEQ ID NO:1 and a light chain variable region comprising SEQ ID NO:2. In some embodiments, the antibody comprises a heavy chain variable region comprising SEQ ID NO:5 and a light chain variable region comprising SEQ ID NO:6. In some embodiments, the antibody comprises a heavy chain variable region comprising SEQ ID NO:9 and a light chain variable region comprising SEQ ID NO:10. In some embodiments, the antibody comprises a heavy chain variable region comprising SEQ ID NO:13 and a light chain variable region comprising SEQ ID NO:14. In some embodiments, the antibody comprises a combination of heavy chain variable regions comprising SEQ ID NO:5, SEQ ID NO:9, and/or SEQ ID NO:13; and light chain variable regions comprising SEQ ID NO:6, SEQ ID NO:10, and/or SEQ ID NO:14. In some embodiments, the antibody comprises a heavy chain variable region comprising SEQ ID NO:1 and a light chain variable region comprising SEQ ID NO:2.

In some embodiments, TrkB agonist antibodies of the invention do not bind to the TrkB ligand binding site and/or compete with BDNF for binding to TrkB. An exemplary antibody that does not bind to the ligand binding site of TrkB is Antibody C20.i.1.1 (also referred to herein as “C20.i1,” “C20.I1,” and “C20”). The heavy chain variable region of antibody C20 is exemplified in SEQ ID NO:1. Accordingly, the invention provides agonist antibodies that compete for binding to TrkB with an antibody comprising a heavy chain variable region comprising SEQ ID NO:1 and a light chain variable region comprising SEQ ID NO:2. In some embodiments, the antibodies of the invention comprise at least one of the complementarity determining regions (CDRs) of SEQ ID NO:1 and/or 2. Without intending to limit the scope of the invention, it is believed that CDR3 plays a significant role in the binding of antibody C20. Accordingly, in some embodiments, an antibody of the present invention comprises SEQ ID NOs: 5 and/or 6. However, CDR1 and/or CDR2 also play a role in binding. Accordingly, in some embodiments, an antibody of the present invention comprises SEQ ID NOs: 9 and/or 10 or 13 and/or 14.

TrkB agonizing antibodies interact with TrkB and are thereby capable of modulating TrkB functions. TrkB agonizing binding molecules can be used to facilitate TrkB pathway signaling; therefore, TrkB agonizing binding molecules can be used to e.g., diagnose, ameliorate the symptoms of, protect against, and treat respiratory disorders associated with aberrantly low levels of TrkB pathway signaling (e.g., due to a mutated version of TrkB or one of its protein interactors in an afflicted subject). Non-limiting examples of disorders associated with aberrant downregulation of TrkB signaling, e.g., due to a mutated version of TrkB or one of its protein interactors, is Rett Syndrome (RTT), which is characterized by mutations in the gene encoding MeCP2 (which binds directly to BDNF).

The present invention also provides methods of treating, diagnosing, preventing, and/or ameliorating respiratory disorders (e.g., Rett Syndrome (RTT)) with pharmaceutical compositions comprising a therapeutically or prophylactically effective amount of the TrkB agonizing antibodies; and a pharmaceutical carrier. In some embodiments, the pharmaceutical composition further comprises a separate and independent agent that is capable of treating, diagnosing, preventing, and/or ameliorating symptoms of respiratory distress (e.g., breathing difficulties), such as small molecule activators of the norepinephrine and/or serotonin pathways (examples are the tricyclic antidepressant desipramine (DMI), the serotonin 1A receptor partial agonist, buspirone, and potentially the more selective antidepressants Fluoxetine and Reboxetine), the activator of glutamatergic AMPA receptors: AMPAkine CX546, prostaglandin, progesterone, or potentiators of TrkB activity (e.g., protein tyrosine phosphatase inhibitors).

Said methods can employ pharmaceutical compositions comprising a therapeutically or prophylactically effective amount of any TrkB agonist antibodies, and those antibodies specifically listed are considered non-limiting embodiments.

The present invention also provides methods of treating, diagnosing, preventing, and/or ameliorating symptoms of respiratory distress, such as those commonly found with respiratory disorders. Said symptoms include but are not limited to breathing difficulties (e.g., stridor or wheezing, breath holding, shallow breathing, hyperventilation, prolonged apneas), poor or decreased oxygenation of the blood (e.g., cyanosis)(e.g., due to impaired absorption of oxygen, inadequate perfusion of the lungs with blood, etc.), and chest pain. In some embodiments, said methods comprise administering a therapeutically or prophylactically effective amount of an antibody agonist of Tyrosine Kinase Receptor B (TrkB) to the individual. In some embodiments, said methods comprise administering a pharmaceutical composition comprising a therapeutically or prophylactically effective amount of TrkB agonizing antibody and a pharmaceutical carrier to the individual. In some embodiments, the individual has one or more respiratory disorders and/or is experiencing one or more symptoms of respiratory distress. In some embodiments, the individual is predisposed to symptoms of respiratory distress. In some embodiments, the individual has Rett Syndrome.

Said methods can employ any TrkB agonist antibodies (or pharmaceutical compositions comprising any TrkB agonist antibodies), and those antibodies specifically listed are considered non-limiting embodiments.

In some embodiments, a therapeutically and/or prophylactically effective amount of a second agent effective in treating, diagnosing, preventing, and/or ameliorating respiratory disorders (e.g., Rett Syndrome (RTT)) is administered to the individual in combination with the antibody agonist of TrkB (or pharmaceutical composition containing the same). In some embodiments, the second agent and the antibody agonist of TrkB (or pharmaceutical composition containing the same) are administered as a mixture. In some embodiments, the second agent is selected from the group consisting of small molecule activators of the norepinephrine and/or serotonin pathways (examples are the tricyclic antidepressant desipramine (DMI), the serotonin 1A receptor partial agonist, buspirone, and potentially the more selective antidepressants Fluoxetine and Reboxetine), the activator of glutamatergic AMPA receptors: AMPAkine CX546, prostaglandin, progesterone, or potentiators of TrkB activity (e.g., protein tyrosine phosphatase inhibitors).

In some embodiments, the antibody is a humanized antibody.

In various embodiments, the invention provides methods of treating, diagnosing, preventing, and/or ameliorating respiratory disorders (e.g., Rett Syndrome (RTT)), or symptoms of respiratory distress, with TrkB agonizing antibodies that modulate (e.g., promote) one or more biological functions of TrkB. For example, a TrkB agonist antibody can modulate dimerization of TrkB, and subsequent auto-phosphorylation of specific tyrosine residues on the TrkB intracellular domain. By way of further example, a TrkB agonist antibody can initiate TrkB-related intracellular signaling cascades (e.g., the mitogen-activated protein kinase, phosphatidylinositol 3-kinase, and PLCγ pathways) that lead to the suppression of neuron death, the promotion of neurite outgrowth, and other effects of the neurotrophins.

TrkB agonizing antibodies include, for example, antibodies that bind to TrkB (e.g., within a particular domain or epitope of TrkB, such to Ligand Binding Domain of TrkB, or outside of the Ligand Binding Domain), and polypeptides that include antigen binding portions of such antibodies. TrkB agonizing antibodies also include molecules in which the binding portion is not derived from an antibody, e.g., TrkB agonizing antibodies derived from polypeptides that have an immunoglobulin-like fold, and in which the antigen binding portion is engineered to bind TrkB through randomization, selection, and affinity maturation.

In various embodiments, the invention provides methods of treating, diagnosing, preventing, and/or ameliorating respiratory disorders (e.g., Rett Syndrome (RTT)), or symptoms of respiratory distress, with TrkB agonist antibodies that bind to an epitope within human TrkB and which are cross reactive with the TrkB protein (or portion thereof) of a non-human primate (e.g., a cynomolgus monkey, or a rhesus monkey). In various embodiments, said TrkB agonist antibody is cross reactive with TrkB of a rodent species (e.g., murine TrkB, rat TrkB). In various embodiments, said TrkB agonist antibody is cross reactive with human TrkA or TrkC.

In other embodiments, the invention provides methods of treating, diagnosing, preventing, and/or ameliorating respiratory disorders (e.g., Rett Syndrome (RTT)), or symptoms of respiratory distress, with TrkB antibodies that binds to an epitope within human TrkB but which are not cross reactive with the TrkB protein (or portion thereof) of a non-human primate (e.g., a cynomolgus monkey, or a rhesus monkey). In various embodiments, said TrkB agonist antibody is not cross reactive with TrkB of a rodent species (e.g., murine TrkB, rat TrkB). In various embodiments, said TrkB agonist antibody does not cross react with human TrkA or TrkC, or with neurotrophin receptor p75NR.

In various embodiments, the antigen binding portion of a TrkB agonizing antibody of the present methods binds to a linear epitope. In various embodiments, said antigen binding portion binds to a non-linear epitope.

In various embodiments, the antigen binding portion of a TrkB agonizing antibody of the present methods binds to TrkB with a dissociation constant (K_{D}) equal to or less than 1 nM, 0.5 nM, 0.25 nM, or 0.1 nM.

In some embodiments, the antibody is capable of binding the human version of TrkB, and not to the TrkB of other species. In some embodiments, the antibody is capable of binding the human version of TrkB, and to the TrkB of other species as well (i.e., is capable of cross-reactivity)(including, e.g., to mouse, rat, and/or non-human primate (e.g., a cynomolgus monkey, or a rhesus monkey)).

In various embodiments, the antigen binding portion of a TrkB agonizing antibody of the present methods binds to TrkB of a non-human primate (e.g., cynomolgus monkey or chimpanzee) with a K_{D} equal to or less than 0.3 nM.

In various embodiments, said antigen binding portion binds to mouse TrkB with a K_{D} equal to or less than 0.5 nM.

In one embodiment, the TrkB agonizing antibody of the present methods is a human antibody. In another embodiment, said TrkB agonist antibody is a non-human antibody. In another embodiment, said TrkB agonist antibody is a chimeric (e.g., humanized, humaneered) antibody.

In one embodiment, the antigen binding portion of a TrkB agonizing antibody of the present methods is an antigen binding portion of a human antibody. Said antigen binding portion can be an antigen binding portion of a monoclonal antibody or a polyclonal antibody.

The TrkB agonizing antibody of the present methods includes, for example, a Fab fragment, a Fab′ fragment, a F(ab′)₂, or an Fv fragment of the antibody.

In some embodiments, the TrkB agonist antibody of the present methods is pegylated. In some embodiments, the TrkB agonist antibody is a pegylated Fab fragment.

In one embodiment, the TrkB agonist antibody of the present methods includes a single chain Fv.

In one embodiment, the TrkB agonist antibody of the present methods includes a diabody (e.g., a single chain diabody, or a diabody having two polypeptide chains).

In some embodiments, the antigen binding portion of the TrkB agonist antibody of the present methods is derived from an antibody of one of the following isotypes: IgG1, IgG2, IgG3 or IgG4. In some embodiments, the antigen binding portion of said antibody is derived from an antibody of an IgA or IgE isotype.

In one embodiment, a TrkB agonist antibody of the present methods competes with BDNF for binding to TrkB, thereby modulating the biological activity and consequences of TrkB pathway signaling. By way of non-limiting example, a TrkB agonist antibody of the present methods can activate, enhance, or perpetuate TrkB pathway activation and signaling (e.g., by competing with BDNF for binding to TrkB). In some embodiments, said TrkB agonist antibody binds to the TrkB Ligand Binding Domain and thereby competes with BDNF for binding to TrkB.

In some embodiments, the antibody of the present methods acts as a BDNF mimetic, and is capable of, e.g., recapitulating the trophic activities of said ligand (and therefore, is capable of exerting neuroprotective and neurotrophic effects).

In other embodiments, the TrkB agonist antibody of the present methods does not bind to the TrkB Ligand Binding Domain, and does not compete with BDNF for binding with TrkB, but is capable nevertheless of modulating the TrkB signaling pathway (e.g., activating, enhancing, or perpetuating TrkB pathway activation and signaling).

In various embodiments, the invention provides methods of treating, diagnosing, preventing, and/or ameliorating respiratory disorders (e.g., Rett Syndrome (RTT)), or symptoms of respiratory distress, with TrkB agonizing antibodies which modulate downstream biological activities normally modulated in a direct or indirect fashion by TrkB. Non-limiting examples of said activities include modulating dimerization of TrkB, and subsequently auto-phosphorylating tyrosine residues on the TrkB intracellular domain; initiating TrkB-related intracellular signaling cascades such as the mitogen-activated protein kinase, phosphatidylinositol 3-kinase, and PLCγ pathways; and suppressing of neuron death, the promotion of neurite outgrowth, and other effects of the neurotrophins. For example, a TrkB agonist antibody of the present methods suppresses neuron death by at least a 5%, 10%, 15%, 25%, or 50%, greater margin relative to a control (e.g., relative to activity in the absence of the TrkB agonist antibody). By way of further example, said TrkB agonist antibody stabilizes TrkB protein levels by at least a 5%, 10%, 15%, 25%, or 50%, greater margin relative to a control (e.g., relative to activity in the absence of the TrkB agonist antibody).

In various embodiments, the invention provides methods of treating, diagnosing, preventing, and/or ameliorating respiratory disorders (e.g., Rett Syndrome (RTT)), or symptoms of respiratory distress, with non-antibody TrkB agonizing molecules. A non-antibody TrkB agonist molecule includes a TrkB binding domain that has an amino acid sequence derived from an immunoglobulin-like (Ig-like) fold of a non-antibody polypeptide, such as one of the following: tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class I MHC, T-cell antigen receptor, CD1, C2 and I-set domains of VCAM-1, I-set immunoglobulin domain of myosin-binding protein C, I-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, or thaumatin. In general, the amino acid sequence of the TrkB binding domain is altered, relative to the amino acid sequence of the immunoglobulin-like fold, such that the TrkB binding domain specifically binds to the TrkB (i.e., wherein the immunoglobulin-like fold does not specifically bind to the TrkB).

In various embodiments, the amino acid sequence of the TrkB binding domain is at least 60% identical (e.g., at least 65%, 75%, 80%, 85%, or 90% identical) to an amino acid sequence of an immunoglobulin-like fold of a fibronectin, a cytokine receptor, or a cadherin.

In various embodiments, the amino acid sequence of the TrkB binding domain is at least 60%, 65%, 75%, 80%, 85%, or 90% identical to an amino acid sequence of an immunoglobulin-like fold of one of the following: tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class I MHC, T-cell antigen receptor, CD1, C2 and I-set domains of VCAM-1, I-set immunoglobulin domain of myosin-binding protein C, I-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, or thaumatin.

In various embodiments, the TrkB binding domain binds to TrkB with a K_{D} equal to or less than 1 nM (e.g., 0.5 nM, 01 nM).

In some embodiments, the Ig-like fold is an Ig-like fold of a fibronectin, e.g., an Ig-like fold of fibronectin type III (e.g., an Ig-like fold of module 10 of fibronectin III).

The invention also features methods of using pharmaceutical compositions that include a TrkB agonist antibody described herein. The composition includes, for example, a TrkB agonist antibody and a pharmaceutically acceptable carrier.

In one aspect, the invention features methods of suppressing neural cell death or promoting neurite outgrowth by administering a therapeutically and/or prophylactically effective amount of an antibody agonist of TrkB (or pharmaceutical composition containing the same). These methods includes contacting tissues or biological samples with a therapeutically and/or prophylactically effective amount of an antibody agonist of TrkB (or pharmaceutical composition containing the same), thereby activating and/or stabilizing the TrkB signaling pathway. The TrkB agonist antibody (or pharmaceutical composition containing the same) can be administered in an amount effective to suppress neural cell death or promote neurite outgrowth.

In some embodiments, the methods feature intra-peritoneal administration of the antibody agonist of TrkB (or pharmaceutical composition containing the same).

Any type of TrkB agonist antibody may be used according to the methods of the invention. Generally, the antibodies used are monoclonal antibodies. Monoclonal antibodies can be generated by any method known in the art (e.g., using hybridomas, recombinant expression, and/or phage display). Without limitation, TrkB agonist antibodies from any of the following patent and non-patent publications can be used in the present methods: Qian, M., et al. (2006) Journal of Neurosci. 26(37); 9394-9403; U.S. Pat. No. 5,910,574 (and any related family members); PCT patent publication number WO06/133164 (and any related family members).

### Definitions

As used herein, the term “respiratory disorders” includes but is not limited to, atelectasis, cystic fibrosis, Rett syndrome (RTT), asthma, apneas (e.g., sleep apnea), acute respiratory distress syndrome (ARDS), chronic obstructive pulmonary disease (COPD), emphysema, acute dyspnea, tachypnea, orthopnea, rheumatoid lung disease, pulmonary congestion or edema, chronic obstructive airway disease (e.g., emphysema, chronic bronchitis, bronchial asthma, and bronchi ectasis), hypoventilation, Pickwickian Syndrome, obesity-hypoventilation syndrome, sudden infant death syndrome (SIDS), and hypercapnea.

Furthermore, “respiratory disorders” also include conditions in humans known to be linked to genetic defects, such as Charcot-Marie-Tooth disease, Cheyne-Stokes breathing disorder, Willi-Prader syndrome, sudden infant death syndrome, congenital central hypoventilation, diffuse interstitial diseases (e.g., sarcoidosis, pneumoconiosis, hypersensitivity pneumonitis, bronchiolitis, Goodpasture's syndrome, idiopathic pulmonary fibrosis, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchiolovlveolar carcinoma, bronchial carcinoid, hamartoma, and mesenchymal tumors).

As used herein, “modulate” indicates the ability to control or influence directly or indirectly, and by way of non-limiting examples, can alternatively mean inhibit or stimulate, agonize or antagonize, hinder or promote, and strengthen or weaken.

A “prophylactically effective dosage,” and a “therapeutically effective dosage,” of TrkB agonizing antibody of the invention can prevent the onset of, or result in a decrease in severity of, respectively, disease symptoms (e.g., symptoms of disorders associated with aberrantly low levels of TrkB, or with mutant copies of TrkB). Said terms can also promote or increase, respectively, frequency and duration of disease symptom-free periods. A “prophylactically effective dosage,” and a “therapeutically effective dosage,” can also prevent or ameliorate, respectively, impairment or disability due to the affliction with TrkB-related or respiratory disorders.

The term “subject” is intended to include organisms, e.g., eukaryotes, which are suffering from or afflicted with a disease, disorder or condition associated with aberrant TrkB signaling pathway. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from respiratory disorders or conditions (e.g., Rett Syndrome (RTT)), as described herein.

The term “antibody” as used herein refers to an intact antibody or an antigen binding fragment (i.e., “antigen-binding portion”) or single chain (i.e., light or heavy chain) thereof. An intact antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen binding portion” of an antibody, as used herein, refers to one or more fragments of an intact antibody that retain the ability to specifically bind to a given antigen (e.g., TrkB). Antigen binding functions of an antibody can be performed by fragments of an intact antibody. Examples of binding fragments encompassed within the term “antigen binding portion” of an antibody include a Fab fragment, a monovalent fragment consisting of the V_{L}, V_{H}, C_{L} and CH1 domains; an F(ab)₂ fragment, a bivalent fragment comprising two Fab fragments (generally one from a heavy chain and one from a light chain) linked by a disulfide bridge at the hinge region; an Fd fragment consisting of the V_{H} and CH1 domains; an Fv fragment consisting of the V_{L} and V_{H} domains of a single arm of an antibody; a single domain antibody (dAb) fragment (Ward et al., 1989 Nature 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by an artificial peptide linker that enables them to be made as a single protein chain in which the V_{L} and V_{H} regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies include one or more “antigen binding portions” of an antibody. These antibody fragments are obtained using conventional techniques known to those of skill in the art, and the fragments are screened for utility in the same manner as are intact antibodies.

Antigen binding portions can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136). Antigen binding portions of antibodies can be grafted into scaffolds based on polypeptides such as Fibronectin type III (Fn3) (see U.S. Pat. No. 6,703,199, which describes fibronectin polypeptide monobodies).

Antigen binding portions can be incorporated into single chain molecules comprising a pair of tandem Fv segments (V_{H}—CH1—V_{H}—CH1) which, together with complementary light chain polypeptides, form a pair of antigen binding regions (Zapata et al., 1995 Protein Eng. 8(10):1057-1062; and U.S. Pat. No. 5,641,870).

The term “camelid antibody,” as used herein, refers to one or more fragments of an intact antibody protein obtained from members of the camel and dromedary (Camelus bactrianus and Calelus dromaderius) family, including New World members such as llama species (Lama paccos, Lama glama and Lama vicugna). A region of the camelid antibody that is the small, single variable domain identified as V_{HH} can be obtained by genetic engineering to yield a small protein having high affinity for a target, resulting in a low molecular weight, antibody-derived protein known as a “camelid nanobody”. See U.S. Pat. No. 5,759,808; see also Stijlemans et al., 2004 J. Biol. Chem. 279: 1256-1261; Dumoulin et al., 2003 Nature 424: 783-788; Pleschberger et al., 2003 Bioconjugate Chem. 14: 440-448; Cortez-Retamozo et al., 2002 Int. J. Cancer 89: 456-62; and Lauwereys. et al., 1998 EMBO J. 17: 3512-3520.

An “isolated TrkB agonist antibody”, as used herein, refers to a binding molecule that is substantially free of molecules having antigenic specificities for antigens other than TrkB (e.g., an isolated antibody that specifically binds TrkB is substantially free of antibodies that specifically bind antigens other than TrkB). An isolated binding molecule that specifically binds TrkB may, however, have cross-reactivity to other antigens, such as TrkB molecules from other species. A binding molecule is “purified” if it is substantially free of cellular material.

As used herein, the term “humaneered antibodies” means antibodies that bind the same epitope but differ in sequence. Example technologies include humaneered antibodies produced by humaneering technology of Kalobios, wherein the sequence of the antigen-binging region is derived by, e.g., mutation, rather than due to conservative amino acid replacements.

As used herein, a TrkB agonist antibody (e.g., an antibody or antigen binding portion thereof) that “specifically binds to TrkB” is intended to refer to a TrkB agonist antibody that binds to TrkB with a K_{D} of 1×10⁻⁷ M or less. A TrkB agonist antibody (e.g., an antibody or antigen binding portion thereof) that “cross-reacts with an antigen” is intended to refer to a TrkB agonist antibody that binds that antigen with a K_{D} of 1×10⁻⁶ M or less. A TrkB agonist antibody (e.g., an antibody or antigen binding portion thereof) that “does not cross-react” with a given antigen is intended to refer to a TrkB agonist antibody that either does not bind detectably to the given antigen, or binds with a K_{D} of 1×10⁻⁵ M or greater. In certain embodiments, such binding molecules that do not cross-react with the antigen exhibit essentially undetectable binding against these proteins in standard binding assays.

The term “monoclonal antibody composition” as used herein refers to a preparation of antibody molecules of single molecular composition. A monoclonal antibody composition displays a single binding specificity and affinity for a particular epitope.

The term “human antibody,” as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to an antibody displaying a single binding specificity that has variable regions in which both the framework and CDR regions are derived from human sequences. In one embodiment, the human monoclonal antibody is produced by a hybridoma that includes a B cell obtained from a transgenic nonhuman animal (e.g., a transgenic mouse having a genome comprising a human heavy chain transgene and a light chain transgene) fused to an immortalized cell.

The term “recombinant human antibody”, as used herein, includes any human antibody that is prepared, expressed, created or isolated by recombinant means, such as an antibody isolated from an animal (e.g., a mouse) that is transgenic or transchromosomal for human immunoglobulin genes or a hybridoma prepared therefrom; an antibody isolated from a host cell transformed to express the human antibody, e.g., from a transfectoma; an antibody isolated from a recombinant, combinatorial human antibody library; and an antibody prepared, expressed, created or isolated by any other means that involve splicing of all or a portion of a human immunoglobulin gene sequences to another DNA sequence. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in a human.

As used herein, “isotype” refers to the antibody class (e.g., IgM, IgE, IgG such as IgG1 or IgG4) that is encoded by the heavy chain constant region gene.

The phrases “an antibody recognizing an antigen” and “an antibody specific for an antigen” are used interchangeably herein with the term “an antibody that binds specifically to an antigen.”

The phrase “specifically (or selectively) binds” to an antibody or is “specifically (or selectively) immunoreactive with,” when referring to a protein or peptide, refers to a binding reaction that is determinative of the presence of the protein in a heterogeneous population of proteins or other biologics. Thus, under designated immunoassay conditions, the specified antibodies bind to a particular protein at least two times the background and do not substantially bind in a significant amount to the other proteins present in the sample. Specific binding to an antibody under such conditions may require an antibody that is selected for its specificity for a particular protein. This selection may be achieved by subtracting out antibodies that cross-react with, e.g., TrkA or TrkC, or neurotrophin receptor p75NR. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1998), for a description of immunoassay formats and conditions that can be used to determine specific immunoreactivity). Typically a specific or selective reaction will be at least twice background signal or noise and more typically more than 10 to 100 times background.

The term “antibody agonist” refers to an antibody capable of activating a receptor to induce a full or partial receptor-mediated response. For example, an agonist of TrkB binds to TrkB and induces TrkB-mediated signaling. In some embodiments, a TrkB antibody against agonist can be identified by its ability to bind TrkB and induce neurite outgrowth when contacted to SH—SY5Y cells or as otherwise described herein.

“Activity” of a polypeptide of the invention refers to structural, regulatory, or biochemical functions of a polypeptide in its native cell or tissue. Examples of activity of a polypeptide include both direct activities and indirect activities. Exemplary direct activities are the result of direct interaction with the polypeptide, including ligand binding, such as binding of BDNF to the Ligand Binding Domain (LBD).

As used herein, the term “high affinity”, when referring to an IgG antibody, indicates that the antibody has a K_{D} of 10⁻⁹ M or less for a target antigen.

A nucleotide sequence is said to be “optimized” if it has been altered to encode an amino acid sequence using codons that are preferred in the production cell or organism, generally a eukaryotic cell, for example, a cell of a yeast such as Pichia, an insect cell, a mammalian cell such as Chinese Hamster Ovary cell (CHO) or a human cell. The optimized nucleotide sequence is engineered to encode an amino acid sequence identical or nearly identical to the amino acid sequence encoded by the original starting nucleotide sequence, which is also known as the “parental” sequence.

Various aspects of the invention are described in further detail in the following subsections.

Standard assays to evaluate the ability of molecules to bind to TrkB of various species, and particular epitopes of TrkB, are known in the art, including, for example, ELISAs and western blots. Determination of whether a TrkB agonist antibody binds to a specific epitope of TrkB can employ a peptide epitope competition assay. For example, a TrkB agonist antibody is incubated with a peptide corresponding to an TrkB epitope of interest at saturating concentrations of peptide. The preincubated TrkB agonist antibody is tested for binding to immobilized TrkB, e.g., by Biacore® analysis. Inhibition of TrkB binding by preincubation with the peptide indicates that the TrkB agonist antibody binds to the peptide epitope (see, e.g., U.S. Pat. Pub. 20070072797). Binding kinetics also can be assessed by standard assays known in the art, such as by Biacore® analysis or apparent binding by FACS analysis. Assays to evaluate the effects of TrkB agonizing antibodies on functional properties of TrkB are described in further detail below.

Accordingly, a TrkB agonist antibody that “inhibits” one or more of these TrkB functional properties (e.g., biochemical, cellular, physiological or other biological activities, or the like), as determined according to methodologies known to the art and described herein, will be understood to produce a statistically significant decrease in the particular functional property relative to that seen in the absence of the binding molecule (e.g., when a control molecule of irrelevant specificity is present). A TrkB agonist antibody that inhibits TrkB activity effects such a statistically significant decrease by at least 5% of the measured parameter. In certain embodiments, an antagonizing antibody or other TrkB agonist antibody may produce a decrease in the selected functional property of at least 10%, 20%, 30%, or 50% compared to control.

A TrkB agonist antibody that agonizes or promotes TrkB activity effectuates such a statistically significant increase by at least 5% of the measured parameter. In certain embodiments, a TrkB agonist antibody or portion thereof may produce a increase in the selected functional property of at least 10%, 20%, 30%, or 50% compared to control.

### Antibodies

The anti-TrkB antibodies described herein include human monoclonal antibodies. In some embodiments, antigen binding portions of antibodies that bind to TrkB, (e.g., V_{H} and V_{L} chains) are “mixed and matched” to create other anti-TrkB agonizing antibodies. The binding of such “mixed and matched” antibodies can be tested using the aforementioned binding assays (e.g., ELISAs). When selecting a V_{H} to mix and match with a particular V_{L} sequence, typically one selects a V_{H} that is structurally similar to the V_{H} it replaces in the pairing with that V_{L}. Likewise a full length heavy chain sequence from a particular full length heavy chain/full length light chain pairing is generally replaced with a structurally similar full length heavy chain sequence. Likewise, a V_{L} sequence from a particular V_{H}/V_{L} pairing should be replaced with a structurally similar V_{L} sequence. Likewise a full length light chain sequence from a particular full length heavy chain/full length light chain pairing should be replaced with a structurally similar full length light chain sequence. Identifying structural similarity in this context is a process well known in the art.

In other aspects, the invention provides antibodies that comprise the heavy chain and light chain CDR1s, CDR2s and CDR3s of one or more TrkB-binding antibodies, in various combinations. Given that each of these antibodies can bind to TrkB and that antigen-binding specificity is provided primarily by the CDR1, 2 and 3 regions, the V_{H} CDR1, 2 and 3 sequences and V_{L} CDR1, 2 and 3 sequences can be “mixed and matched” (i.e., CDRs from different antibodies can be mixed and matched). TrkB binding of such “mixed and matched” antibodies can be tested using the binding assays described herein (e.g., ELISAs). When V_{H} CDR sequences are mixed and matched, the CDR1, CDR2 and/or CDR3 sequence from a particular V_{H} sequence should be replaced with a structurally similar CDR sequence(s). Likewise, when V_{L} CDR sequences are mixed and matched, the CDR1, CDR2 and/or CDR3 sequence from a particular V_{L} sequence should be replaced with a structurally similar CDR sequence(s). Identifying structural similarity in this context is a process well known in the art.

As used herein, a human antibody comprises heavy or light chain variable regions or full length heavy or light chains that are “the product of” or “derived from” a particular germline sequence if the variable regions or full length chains of the antibody are obtained from a system that uses human germline immunoglobulin genes as the source of the sequences. In one such system, a human antibody is raised in a transgenic mouse carrying human immunoglobulin genes. The transgenic is immunized with the antigen of interest (e.g., an epitope of TrkB). Alternatively, a human antibody is identified by providing a human immunoglobulin gene library displayed on phage and screening the library with the antigen of interest (e.g., an epitope of TrkB).

A human antibody that is “the product of or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of or “derived from” a particular human germline immunoglobulin sequence may contain amino acid differences as compared to the germline-encoded sequence, due to, for example, naturally occurring somatic mutations or artificial site-directed mutations. However, a selected human antibody typically has an amino acid sequence at least 90% identical to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene.

The percent identity between two sequences is a function of the number of identity positions shared by the sequences (i.e., % identity=# of identity positions/total # of positions×100), taking into account the number of gaps, and the length of each gap, that need to be introduced for optimal alignment of the two sequences. The comparison of sequences and determination of percent identity between two sequences is determined using the algorithm of E. Meyers and W. Miller (1988 Comput. Appl. Biosci., 4:11-17) which has been incorporated into the ALIGN program (version 2.0), using a PAM120 weight residue table, a gap length penalty of 12 and a gap penalty of 4.

Typically, a V_{H} or V_{L} of a human antibody derived from a particular human germline sequence will display no more than 10 amino acid differences from the amino acid sequence encoded by the human germline immunoglobulin gene. In certain cases, the V_{H} or V_{L} of the human antibody may display no more than 5, or even no more than 4, 3, 2, or 1 amino acid difference from the amino acid sequence encoded by the germline immunoglobulin gene.

### Camelid Antibodies

Antibody proteins obtained from members of the camel and dromedary (Camelus bactrianus and Calelus dromaderius) family, including New World members such as llama species (Lama paccos, Lama glama and Lama vicugna), have been characterized with respect to size, structural complexity and antigenicity for human subjects. Certain IgG antibodies found in nature in this family of mammals lack light chains, and are thus structurally distinct from the four chain quaternary structure having two heavy and two light chains typical for antibodies from other animals. See WO 94/04678.

A region of the camelid antibody that is the small, single variable domain identified as V_{HH} can be obtained by genetic engineering to yield a small protein having high affinity for a target, resulting in a low molecular weight, antibody-derived protein known as a “camelid nanobody”. See U.S. Pat. No. 5,759,808; see also Stijlemans et al., 2004 J. Biol. Chem. 279: 1256-1261; Dumoulin et al., 2003 Nature 424: 783-788; Pleschberger et al., 2003 Bioconjugate Chem. 14: 440-448; Cortez-Retamozo et al., 2002 Int. J. Cancer 89: 456-62; and Lauwereys. et al., 1998 EMBO J. 17: 3512-3520. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents to detect antigens that are otherwise cryptic using classical immunological techniques, and as possible therapeutic agents. Thus, yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies' being extremely thermostable, stable to extreme pH and to proteolytic digestion, and poorly antigenic. Another consequence is that camelid nanobodies readily move from the circulatory system into tissues, and even cross the blood-brain barrier and can treat disorders that affect nervous tissue. Nanobodies can further facilitate drug transport across the blood brain barrier. See U.S. Pat. Pub. No. 20040161738, published Aug. 19, 2004. These features combined with the low antigenicity in humans indicate great therapeutic potential. Further, these molecules can be fully expressed in prokaryotic cells such as E. coli.

Accordingly, a feature of the present invention is a camelid antibody or camelid nanobody having high affinity for TrkB. In certain embodiments herein, the camelid antibody or nanobody is naturally produced in the camelid animal, i.e., is produced by the camelid following immunization with TrkB or a peptide fragment thereof, using techniques described herein for other antibodies. Alternatively, the anti-TrkB camelid nanobody is engineered, i.e., produced by selection, for example from a library of phage displaying appropriately mutagenized camelid nanobody proteins using panning procedures with TrkB or an TrkB epitope described herein as a target. Engineered nanobodies can further be customized by genetic engineering to increase the half life in a recipient subject from 45 minutes to two weeks.

### Diabodies

Diabodies are bivalent, bispecific molecules in which V_{H} and V_{L} domains are expressed on a single polypeptide chain, connected by a linker that is too short to allow for pairing between the two domains on the same chain. The V_{H} and V_{L} domains pair with complementary domains of another chain, thereby creating two antigen binding sites (see e.g., Holliger et al., 1993 Proc. Natl. Acad. Sci. USA 90:6444-6448; Poljak et al., 1994 Structure 2:1121-1123). Diabodies can be produced by expressing two polypeptide chains with either the structure V_{HA}—V_{LB} and V_{HB}—V_{LA} (V_{H}—V_{L} configuration), or V_{LA}—V_{HB} and V_{LB}—V_{HA} (V_{L}—V_{H} configuration) within the same cell. Most of them can be expressed in soluble form in bacteria.

Single chain diabodies (scDb) are produced by connecting the two diabody-forming polypeptide chains with linker of approximately 15 amino acid residues (see Holliger and Winter, 1997 Cancer Immunol. Immunother., 45(3-4):128-30; Wu et al., 1996 Immunotechnology, 2(1):21-36). scDb can be expressed in bacteria in soluble, active monomeric form (see Holliger and Winter, 1997 Cancer Immunol. Immunother., 45(34): 128-30; Wu et al., 1996 Immunotechnology, 2(1):21-36; Pluckthun and Pack, 1997 Immunotechnology, 3(2): 83-105; Ridgway et al., 1996 Protein Eng., 9(7):617-21).

A diabody can be fused to Fc to generate a “di-diabody” (see Lu et al., 2004 J. Biol. Chem., 279(4):2856-65).

### Engineered and Modified Antibodies

An antibody of the invention can be prepared using an antibody having one or more V_{H} and/or V_{L} sequences as starting material to engineer a modified antibody, which modified antibody may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain CDRs. For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al., 1998 Nature 332:323-327; Jones et al., 1986 Nature 321:522-525; Queen et al., 1989 Proc. Natl. Acad. See. U.S.A. 86:10029-10033; U.S. Pat. No. 5,225,539, and U.S. Pat. Nos. 5,530,101; 5,585,089; 5,693,762 and 6,180,370).

Framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat et al., 1991 Sequences of Proteins of Immunological Interest, Fifth Edition, U.S. Department of Health and Human Services, NIH Publication No. 91-3242; Tomlinson et al., 1992 J. Mol. Biol. 227:776-798; and Cox et al., 1994 Eur. J. Immunol. 24:827-836; the contents of each of which are expressly incorporated herein by reference.

The V_{H} CDR1, 2 and 3 sequences and the V_{L} CDR1, 2 and 3 sequences can be grafted onto framework regions that have the identical sequence as that found in the germline immunoglobulin gene from which the framework sequence is derived, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370).

CDRs can also be grafted into framework regions of polypeptides other than immunoglobulin domains. Appropriate scaffolds form a conformationally stable framework that displays the grafted residues such that they form a localized surface and bind the target of interest (e.g., TrkB). For example, CDRs can be grafted onto a scaffold in which the framework regions are based on fibronectin, ankyrin, lipocalin, neocarzinostain, cytochrome b, CP1 zinc finger, PST1, coiled coil, LACI-D1, Z domain or tendramisat (See e.g., Nygren and Uhlen, 1997 Current Opinion in Structural Biology, 7, 463-469).

Another type of variable region modification is mutation of amino acid residues within the V_{H} and/or V_{L} CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s), and the effect on antibody binding, or other functional property of interest, can be evaluated in in vitro or in vivo assays as described herein. Conservative modifications can be introduced. The mutations may be amino acid substitutions, additions or deletions. Moreover, typically no more than one, two, three, four or five residues within a CDR region are altered.

Engineered antibodies of the invention include those in which modifications have been made to framework residues within V_{H} and/or V_{L}, e.g., to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed by the invention.

Another type of framework modification involves mutating one or more residues within the framework region, or even within one or more CDR regions, to remove T cell—epitopes to thereby reduce the potential immunogenicity of the antibody. This approach is also referred to as “deimmunization” and is described in further detail in U.S. Pat. Pub. No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2—CH3 domain interface region of the Fe-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, U.S. Pat. No. 6,277,375 describes the following mutations in an IgG that increase its half-life in vivo: T252L, T254S, T256F. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered C1q binding and/or reduced or abolished complement dependent cytotoxicity (CDC). This approach is described in further detail in U.S. Pat. Nos. 6,194,551 by Idusogie et al.

In another embodiment, one or more amino acid residues are altered to thereby alter the ability of the antibody to fix complement. This approach is described further in WO 94/29351 by Bodmer et al.

In yet another embodiment, the Fc region is modified to increase the ability of the antibody to mediate antibody dependent cellular cytotoxicity (ADCC) and/or to increase the affinity of the antibody for an Fcγ receptor by modifying one or more amino acids. This approach is described further in WO 00/42072 by Presta. Moreover, the binding sites on human IgG1 for FcγR1, FcγRII, FcγRIII and FcRn have been mapped and variants with improved binding have been described (see Shields, R. L. et al., 2001 J. Biol. Chem. 276:6591-6604).

In still another embodiment, the glycosylation of an antibody is modified. For example, an aglycoslated antibody can be made (i.e., the antibody lacks glycosylation). Glycosylation can be altered, for example, to increase the affinity of the antibody for an antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Pub. WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180).

Another modification of the antibodies herein that is contemplated by the invention is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG moieties become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al.

In addition, pegylation can be achieved in any part of an TrkB binding polypeptide of the invention by the introduction of a nonnatural amino acid. Certain nonnatural amino acids can be introduced by the technology described in Deiters et al., J Am Chem Soc 125:11782-11783, 2003; Wang and Schultz, Science 301:964-967, 2003; Wang et al., Science 292:498-500, 2001; Zhang et al., Science 303:371-373, 2004 or in U.S. Pat. No. 7,083,970. Briefly, some of these expression systems involve site-directed mutagenesis to introduce a nonsense codon, such as an amber TAG, into the open reading frame encoding a polypeptide of the invention. Such expression vectors are then introduced into a host that can utilize a tRNA specific for the introduced nonsense codon and charged with the nonnatural amino acid of choice. Particular nonnatural amino acids that are beneficial for purpose of conjugating moieties to the polypeptides of the invention include those with acetylene and azido side chains. The polypeptides containing these novel amino acids can then be pegylated at these chosen sites in the protein.

### Methods of Engineering Antibodies

As discussed above, anti-TrkB antibodies can be used to create new anti-TrkB antibodies by modifying full length heavy chain and/or light chain sequences, V_{H} and/or V_{L} sequences, or the constant region(s) attached thereto. For example, one or more CDR regions of the antibodies can be combined recombinantly with known framework regions and/or other CDRs to create new, recombinantly-engineered, anti-TrkB antibodies. Other types of modifications include those described in the previous section. The starting material for the engineering method is one or more of the V_{H} and/or V_{L} sequences, or one or more CDR regions thereof. To create the engineered antibody, it is not necessary to actually prepare (i.e., express as a protein) an antibody having one or more of the V_{H} and/or V_{L} sequences, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Standard molecular biology techniques can be used to prepare and express the altered antibody sequence. The antibody encoded by the altered antibody sequence(s) is one that retains one, some or all of the functional properties of the anti-TrkB antibody from which it is derived, which functional properties include, but are not limited to, specifically binding to TrkB, interfering with TrkB's ability to bind neurotrophins (e.g., BDNF), and modulating TrkB's ability to dimerize and auto-phosphorylate tyrosine residues on its intracellular domain, as described herein. The functional properties of the altered antibodies can be assessed using standard assays available in the art and/or described herein (e.g., ELISAs).

In certain embodiments of the methods of engineering antibodies of the invention, mutations can be introduced randomly or selectively along all or part of an anti-TrkB antibody coding sequence and the resulting modified anti-TrkB antibodies can be screened for binding activity and/or other functional properties (e.g., specifically binding to TrkB, interfering with TrkB's ability to bind neurotrophins (e.g., BDNF), and modulating TrkB's ability to dimerize and auto-phosphorylate tyrosine residues on its intracellular domain, as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, WO 03/074679 by Lazar et al. describes methods of using computational screening methods to optimize physiochemical properties of antibodies.

### Non-Antibody TrkB Agonizing Antibodies

The invention further provides TrkB agonizing antibodies that exhibit functional properties of antibodies but derive their framework and antigen binding portions from other polypeptides (e.g., polypeptides other than those encoded by antibody genes or generated by the recombination of antibody genes in vivo). The antigen binding domains (e.g., TrkB binding domains) of these binding molecules are generated through a directed evolution process. See U.S. Pat. No. 7,115,396. Molecules that have an overall fold similar to that of a variable domain of an antibody (an “immunoglobulin-like” fold) are appropriate scaffold proteins. Scaffold proteins suitable for deriving antigen binding molecules include fibronectin or a fibronectin dimer, tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class I MHC, T-cell antigen receptor, CD1, C2 and I-set domains of VCAM-1, I-set immunoglobulin domain of myosin-binding protein C, I-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, and thaumatin.

The antigen binding domain (e.g., the immunoglobulin-like fold) of the non-antibody binding molecule can have a molecular mass less than 10 kD or greater than 7.5 kD (e.g., a molecular mass between 7.5-10 kD). The protein used to derive the antigen binding domain is a naturally occurring mammalian protein (e.g., a human protein), and the antigen binding domain includes up to 50% (e.g., up to 34%, 25%, 20%, or 15%), mutated amino acids as compared to the immunoglobulin-like fold of the protein from which it is derived. The domain having the immunoglobulin-like fold generally consists of 50-150 amino acids (e.g., 40-60 amino acids).

To generate non-antibody binding molecules, a library of clones is created in which sequences in regions of the scaffold protein that form antigen binding surfaces (e.g., regions analogous in position and structure to CDRs of an antibody variable domain immunoglobulin fold) are randomized. Library clones are tested for specific binding to the antigen of interest (e.g., TrkB) and for other functions (e.g., inhibition of biological activity of TrkB). Selected clones can be used as the basis for further randomization and selection to produce derivatives of higher affinity for the antigen. One example of a selection protocol is described in U.S. Pat. No. 6,207,446.

High affinity binding molecules are generated, for example, using the tenth module of fibronectin III (¹⁰Fn3) as the scaffold. A library is constructed for each of three CDR-like loops of ¹⁰FN3 at residues 23-29, 52-55, and 78-87. To construct each library, DNA segments encoding sequence overlapping each CDR-like region are randomized by oligonucleotide synthesis. Techniques for producing selectable ¹⁰Fn3 libraries are described in U.S. Pat. Nos. 6,818,418 and 7,115,396; Roberts and Szostak, 1997 Proc. Natl. Acad. Sci USA 94:12297; U.S. Pat. No. 6,261,804; U.S. Pat. No. 6,258,558; and Szostak et al. WO98/31700.

Non-antibody binding molecules can be produces as dimers or multimers to increase avidity for the target antigen. For example, the antigen binding domain is expressed as a fusion with a constant region (Fc) of an antibody that forms Fc-Fc dimers. See, e.g., U.S. Pat. No. 7,115,396.

### Nucleic Acid Molecules Encoding Antibodies of the Invention

Another aspect of the invention pertains to nucleic acid molecules that encode the TrkB agonizing antibodies of the invention. The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. 1987 Current Protocols in Molecular Biology, Greene Publishing and Wiley Interscience, New York. A nucleic acid of the invention can be, for example, DNA or RNA and may or may not contain intronic sequences. In an embodiment, the nucleic acid is a cDNA molecule. The nucleic acid may be present in a vector such as a phage display vector, or in a recombinant plasmid vector.

Nucleic acids of the invention can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from various phage clones that are members of the library.

Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to an scFv gene. In these manipulations, a V_{L}- or V_{H}-encoding DNA fragment is operatively linked to another DNA molecule, or to a fragment encoding another protein, such as an antibody constant region or a flexible linker. The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter.

The isolated DNA encoding the V_{H} region can be converted to a full-length heavy chain gene by operatively linking the V_{H}-encoding DNA to another DNA molecule encoding heavy chain constant regions (CH1, CH2 and CH3). The sequences of human heavy chain constant region genes are known in the art (see e.g., Kabat et al., 1991 Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region. For a Fab fragment heavy chain gene, the V_{H}-encoding DNA can be operatively linked to another DNA molecule encoding only the heavy chain CH1 constant region.

The isolated DNA encoding the V_{L} region can be converted to a full-length light chain gene (as well as to a Fab light chain gene) by operatively linking the V_{L}-encoding DNA to another DNA molecule encoding the light chain constant region, CL. The sequences of human light chain constant region genes are known in the art (see e.g., Kabat et al., 1991 Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or a lambda constant region.

To create an scFv gene, the V_{H}- and V_{L}-encoding DNA fragments are operatively linked to another fragment encoding a flexible linker, e.g., encoding the amino acid sequence (Gly4 -Ser)₃, such that the V_{H} and V_{L} sequences can be expressed as a contiguous single-chain protein, with the V_{L} and V_{H} regions joined by the flexible linker (see e.g., Bird et al., 1988 Science 242:423-426; Huston et al., 1988 Proc. Natl. Acad. Sci. USA 85:5879-5883; McCafferty et al., 1990 Nature 348:552-554).

### Monoclonal Antibody Generation

Monoclonal antibodies (mAbs) can be produced by a variety of techniques, including conventional monoclonal antibody methodology e.g., the standard somatic cell hybridization technique of Kohler and Milstein (1975 Nature, 256:495), or using library display methods, such as phage display.

An animal system for preparing hybridomas is the murine system. Hybridoma production in the mouse is a well established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539, and U.S. Pat. Nos. 5,530,101; 5,585,089; 5,693,762 and 6,180,370.

In a certain embodiment, the antibodies of the invention are human monoclonal antibodies. Such human monoclonal antibodies directed against TrkB can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HuMAb mouse® (Medarex, Inc.) contains human immunoglobulin gene miniloci that encode un-rearranged human heavy (μ and γ) and κ light chain immunoglobulin sequences, together with targeted mutations that inactivate the endogenous μ and κ chain loci (see, e.g., Lonberg et al., 1994 Nature 368(6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg, N. et al., 1994 supra; reviewed in Lonberg, N., 1994 Handbook of Experimental Pharmacology 113:49-101; Lonberg, N. and Huszar, D., 1995 Intern. Rev. Immunol.13: 65-93, and Harding, F. and Lonberg, N., 1995 Ann. N. Y. Acad. Sci. 764:536-546). The preparation and use of HuMAb mice, and the genomic modifications carried by such mice, is further described in Taylor, L. et al., 1992 Nucleic Acids Research 20:6287-6295; Chen, J. et at., 1993 International Immunology 5: 647-656; Tuaillon et al., 1993 Proc. Natl. Acad. Sci. USA 94:3720-3724; Choi et al., 1993 Nature Genetics 4:117-123; Chen, J. et al., 1993 EMBO J. 12: 821-830; Tuaillon et al., 1994 J. Immunol. 152:2912-2920; Taylor, L. et al., 1994 International Immunology 579-591; and Fishwild, D. et al., 1996 Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Pub. Nos. WO 92103918, WO 93/12227, WO 94/25585, WO 97113852, WO 98/24884 and WO 99/45962, all to Lonberg and Kay; and PCT Pub. No. WO 01/14424 to Korman et al.

In another embodiment, human antibodies of the invention can be raised using a mouse that carries human immunoglobulin sequences on transgenes and transchomosomes, such as a mouse that carries a human heavy chain transgene and a human light chain transchromosome. Such mice, referred to herein as “KM mice”, are described in detail in WO 02/43478.

Still further, alternative transgenic animal systems expressing human immunoglobulin genes are available in the art and can be used to raise anti-TrkB antibodies of the invention. For example, an alternative transgenic system referred to as the Xenomouse® (Abgenix, Inc.) can be used. Such mice are described in, e.g., U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise anti-TrkB antibodies of the invention. For example, mice carrying both a human heavy chain transchromosome and a human light chain tranchromosome, referred to as “TC mice” can be used; such mice are described in Tomizuka et al., 2000 Proc. Natl. Acad. Sci. USA 97:722-727. Furthermore, cows carrying human heavy and light chain transchromosomes have been described in the art (Kuroiwa et al., 2002 Nature Biotechnology 20:889-894) and can be used to raise anti-TrkB antibodies of the invention.

Human monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al. Libraries can be screened for binding to full length TrkB or to a particular epitope of TrkB.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

### Generation of Human Monoclonal Antibodies in Human Ig Mice

Purified recombinant human TrkB expressed in prokaryotic cells (e.g., E. coli) or eukaryotic cells (e.g., mammalian cells, e.g., HEK293 cells) can be used as the antigen. The protein can be conjugated to a carrier, such as keyhole limpet hemocyanin (KLH).

Fully human monoclonal antibodies to TrkB are prepared using HCo7, HCo12 and HCo17 strains of HuMab transgenic mice and the KM strain of transgenic transchromosomic mice, each of which express human antibody genes. In each of these mouse strains, the endogenous mouse kappa light chain gene can be homozygously disrupted as described in Chen et al., 1993 EMBO J. 12:811-820 and the endogenous mouse heavy chain gene can be homozygously disrupted as described in Example 1 of WO 01109187. Each of these mouse strains carries a human kappa light chain transgene, KCo5, as described in Fishwild et al., 1996 Nature Biotechnology 14:845-851. The HCo7 strain carries the HCo7 human heavy chain transgene as described in U.S. Pat. Nos. 5,545,806; 5,625,825; and 5,545,807. The HCo12 strain carries the HCo12 human heavy chain transgene as described in Example 2 of WO 01/09187. The HCo17 stain carries the HCo17 human heavy chain transgene. The KNM strain contains the SC20 transchromosome as described in WO 02/43478.

To generate fully human monoclonal antibodies to TrkB, HuMab mice and KM mice are immunized with purified recombinant TrkB, an TrkB fragment, or a conjugate thereof (e.g., TrkB-KLH) as antigen. General immunization schemes for HuMab mice are described in Lonberg, N. et al., 1994 Nature 368(6474): 856-859; Fishwild, D. et al., 1996 Nature Biotechnology 14:845-851 and WO 98/24884. The mice are 6-16 weeks of age upon the first infusion of antigen. A purified recombinant preparation (5-50 μg) of the antigen is used to immunize the HuMab mice and KM mice in the peritoneal cavity, subcutaneously (Sc) or by footpad injection.

Transgenic mice are immunized twice with antigen in complete Freund's adjuvant or Ribi adjuvant either in the peritoneal cavity (IP), subcutaneously (Sc) or by footpad (FP), followed by 3-21 days IP, Sc or FP immunization (up to a total of 11 immunizations) with the antigen in incomplete Freund's or Ribi adjuvant. The immune response is monitored by retroorbital bleeds. The plasma is screened by ELISA, and mice with sufficient titers of anti-TrkB human immunogolobulin are used for fusions. Mice are boosted intravenously with antigen 3 and 2 days before sacrifice and removal of the spleen. Typically, 10-35 fusions for each antigen are performed. Several dozen mice are immunized for each antigen. A total of 82 mice of the HCo7, HCo12, HCo17 and KM mice strains are immunized with TrkB.

To select HuMab or KM mice producing antibodies that bound TrkB, sera from immunized mice can be tested by ELISA as described by Fishwild, D. et al., 1996. Briefly, microtiter plates are coated with purified recombinant TrkB at 1-2 μg/ml in PBS, 50 μl/wells incubated 4° C. overnight then blocked with 200 μl/well of 5% chicken serum in PBS/Tween (0.05%). Dilutions of plasma from TrkB-immunized mice are added to each well and incubated for 1-2 hours at ambient temperature. The plates are washed with PBS/Tween and then incubated with a goat-anti-human IgG Fc polyclonal antibody conjugated with horseradish peroxidase (HRP) for 1 hour at room temperature. After washing, the plates are developed with ABTS substrate (Sigma, A-1888, 0.22 mg/ml) and analyzed by spectrophotometer at OD 415-495. Splenocytes of mice that developed the highest titers of anti-TrkB antibodies are used for fusions. Fusions are performed and hybridoma supernatants are tested for anti-TrkB activity by ELISA.

The mouse splenocytes, isolated from the HuMab mice and KM mice, are fused with PEG to a mouse myeloma cell line based upon standard protocols. The resulting hybridomas are then screened for the production of antigen-specific antibodies. Single cell suspensions of splenic lymphocytes from immunized mice are fused to one-fourth the number of SP2/0 nonsecreting mouse myeloma cells (ATCC, CRL 1581) with 50% PEG (Sigma). Cells are plated at approximately 1×10⁵/well in flat bottom microtiter plates, followed by about two weeks of incubation in selective medium containing 10% fetal bovine serum, 10% P388D 1(ATCC, CRL TIB-63) conditioned medium, 3-5% Origen® (IGEN) in DMEM (Mediatech, CRL 10013, with high glucose, L-glutamine and sodium pyruvate) plus 5 mM HEPES, 0.055 mM 2-mercaptoethanol, 50 μg/ml gentamycin and 1× HAT (Sigma, CRL P-7185). After 1-2 weeks, cells are cultured in medium in which the HAT is replaced with HT. Individual wells are then screened by ELISA for human anti-TrkB monoclonal IgG antibodies. Once extensive hybridoma growth occurred, medium is monitored usually after 10-14 days. The antibody secreting hybridomas are replated, screened again and, if still positive for human IgG, anti-TrkB monoclonal antibodies are subcloned at least twice by limiting dilution. The stable subclones are then cultured in vitro to generate small amounts of antibody in tissue culture medium for further characterization.

### Generation of Hybridomas Producing Human Monoclonal Antibodies

To generate hybridomas producing human monoclonal antibodies of the invention, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3X63-Ag8.653 nonsecreting mouse myeloma cells (ATCC, CRL 1580) with 50% PEG. Cells are plated at approximately 2×145 in flat bottom microtiter plates, followed by a two week incubation in selective medium containing 20% fetal Clone Serum, 18% “653” conditioned media, 5% Origen® (IGEN), 4 mM L-glutamine, 1 mM sodium pyruvate, 5 mM HEPES, 0:055 mM 2-mercaptoethanol, 50 units/ml penicillin, 50 μg/ml streptomycin, 50 μg/ml gentamycin and 1× HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD₂₈₀ using an extinction coefficient of 1.43. The monoclonal antibodies can be aliquoted and stored at −80° C.

### Generation of Transfectomas Producing Monoclonal Antibodies

Antibodies of the invention also can be produced in a host cell transfectoma using, for example, a combination of recombinant DNA techniques and gene transfection methods as is well known in the art (e.g., Morrison, 1985 Science 229:1202).

For example, to express the antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the CH segment(s) within the vector and the V_{L} segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. 1990 Methods in Enzymology 185, Academic Press, San Diego, Calif.). It will be appreciated by those skilled in the art that the design of the expression vector, including the selection of regulatory sequences, may depend on such factors as the choice of the host cell to be transformed, the level of expression of protein desired, etc. Regulatory sequences for mammalian host cell expression include viral elements that direct high levels of protein expression in mammalian cells, such as promoters and/or enhancers derived from cytomegalovirus (CMV), Simian Virus 40 (SV40), adenovirus (e.g., the adenovirus major late promoter (AdMLP)), and polyoma. Alternatively, nonviral regulatory sequences may be used, such as the ubiquitin promoter or P-globin promoter. Still further, regulatory elements composed of sequences from different sources, such as the SRa promoter system, which contains sequences from the SV40 early promoter and the long terminal repeat of human T cell leukemia virus type 1 (Takebe et al., 1988 Mol. Cell. Biol. 8:466-472).

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665; and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr- host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is theoretically possible to express the antibodies of the invention in either prokaryotic or eukaryotic host cells. Expression of antibodies in eukaryotic cells, in particular mammalian host cells, is discussed because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss and Wood, 1985 Immunology Today 6:12-13).

Mammalian host cells for expressing the recombinant antibodies of the invention include Chinese Hamster Ovary (CHO cells) (including dhfr-CHO cells, described Urlaub and Chasin, 1980 Proc. Natl. Acad. Sci. USA 77:4216-4220 used with a DH FR selectable marker, e.g., as described in Kaufman and Sharp, 1982 Mol. Biol. 159:601-621, NSO myeloma cells, COS cells and SP2 cells. In particular, for use with NSO myeloma cells, another expression system is the GS gene expression system shown in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods.

### Bispecific Molecules

In another aspect, the present invention features bispecific molecules comprising a TrkB agonist antibody (e.g., an anti-TrkB antibody, or a fragment thereof), of the invention. A TrkB agonizing antibody of the invention can be derivatized or linked to another functional molecule, e.g., another peptide or protein (e.g., another antibody or ligand for a receptor) to generate a bispecific molecule that binds to at least two different binding sites or target molecules. The TrkB agonizing antibody of the invention may in fact be derivatized or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present invention includes bispecific molecules comprising at least one first binding specificity for TrkB and a second binding specificity for a second target epitope.

In one embodiment, the bispecific molecules of the invention comprise as a binding specificity at least one antibody, or an antibody fragment thereof, including, e.g., an Fab, Fab′, F(ab′)₂, Fv, or a single chain Fv. The antibody may also be a light chain or heavy chain dimer, or any minimal fragment thereof such as a Fv or a single chain construct as described in Ladner et al. U.S. Pat. No. 4,946,778, the contents of which is expressly incorporated by reference.

The bispecific molecules of the present invention can be prepared by conjugating the constituent binding specificities using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-1-carboxylate (sulfo-SMCC) (see e.g., Karpovsky et al., 1984 J. Exp. Med. 160:1686; Liu et al., 1985 Proc. Natl. Acad. Sci. USA 82:8648). Other methods include those described in Paulus, 1985 Behring Ins. Mitt. No. 78, 118-132; Brennan et al., 1985 Science 229:81-83), and Glennie et al., 1987 J. Immunol. 139: 2367-2375). Conjugating agents are SATA and sulfo-SMCC, both available from Pierce Chemical Co. (Rockford, Ill.).

When the binding specificities are antibodies, they can be conjugated by sulfhydryl bonding of the C-terminus hinge regions of the two heavy chains. In a particular embodiment, the hinge region is modified to contain an odd number of sulfhydryl residues, for example one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)₂ or ligand×Fab fusion protein. A bispecific molecule of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the bispecific molecules to their specific targets can be confirmed by, for example, enzyme-linked immunosorbent assay (ELISA), radioimmunoassay (REA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

### Pharmaceutical Compositions

In another aspect, the present invention provides a composition, e.g., a pharmaceutical composition, containing one or a combination of TrkB agonizing antibodies (e.g., monoclonal antibodies, or antigen-binding portion(s) thereof), of the present invention, formulated together with a pharmaceutically acceptable carrier. Such compositions may include one or a combination of (e.g., two or more different) binding molecules. For example, a pharmaceutical composition of the invention can comprise a combination of antibodies or agents that bind to different epitopes on the target antigen or that have complementary activities.

Pharmaceutical compositions of the invention also can be administered in combination therapy, i.e., combined with other agents. For example, for the treatment of respiratory disorders, the combination therapy can include a TrkB agonist antibody combined with at least one other agent. Examples of therapeutic agents that can be used in combination therapy include but are not limited to small molecule activators of the norepinephrine and/or serotonin pathways (examples are the tricyclic antidepressant desipramine (DMI), the serotonin 1A receptor partial agonist, buspirone, and potentially the more selective antidepressants Fluoxetine and Reboxetine), prostaglandin, progesterone, or potentiators of TrkB activity (e.g., protein tyrosine phosphatase inhibitors).

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for oral, intra-peritoneal, intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al., 1977 J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and di-carboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 per cent to about ninety-nine percent of active ingredient, from about 0.1 per cent to about 70 per cent, or from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals.

An exemplary treatment regime entails administration twice a week, once a week, once every two weeks or once a month. Dosage regimens for TrkB agonizing antibodies of the invention include 1 mg/kg body weight or 3 mg/kg body weight by intra-peritoneal administration, with the antibody being given using one of the following dosing schedules: 1 mg/kg body weight once a week for four weeks, followed by 3 mg/kg body weight once a week for the remaining period of treatment, for example.

In some methods, two or more binding molecules (e.g., monoclonal antibodies) with different binding specificities are administered simultaneously, in which case the dosage of each antibody administered falls within the ranges indicated. The TrkB agonist antibody is usually administered on multiple occasions. Intervals between single dosages can be, for example, weekly, monthly, every three months or yearly. Intervals can also be irregular as indicated by measuring blood levels of binding molecule to TrkB in the patient. In some methods, dosage is adjusted to achieve the proper plasma concentration of the TrkB agonist antibody.

Alternatively, a TrkB agonist antibody can be administered as a sustained release formulation, in which case less frequent administration is required. Dosage and frequency vary depending on the half-life of the TrkB agonist antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated or until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A composition of the present invention can be administered by one or more routes of administration using one or more of a variety of methods known in the art. As will be appreciated by the skilled artisan, the route and/or mode of administration will vary depending upon the desired results. Routes of administration for TrkB agonizing antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

Alternatively, an TrkB agonizing antibody of the invention can be administered by a nonparenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in one embodiment, a therapeutic composition of the invention can be administered with a needleless hypodermic injection device, such as the devices shown in U.S. Pat. Nos. 5,399,163; 5,383,851; 5,312,335; 5,064,413; 4,941,880; 4,790,824 or 4,596,556. Examples of well known implants and modules useful in the present invention include: U.S. Pat. No. 4,487,603, which shows an implantable micro-infusion pump for dispensing medication at a controlled rate; U.S. Pat. No. 4,486,194, which shows a therapeutic device for administering medicants through the skin; U.S. Pat. No. 4,447,233, which shows a medication infusion pump for delivering medication at a precise infusion rate; U.S. Pat. No. 4,447,224, which shows a variable flow implantable infusion apparatus for continuous drug delivery; U.S. Pat. No. 4,439,196, which shows an osmotic drug delivery system having multi-chamber compartments; and U.S. Pat. No. 4,475,196, which shows an osmotic drug delivery system. These patents are incorporated herein by reference. Many other such implants, delivery systems, and modules are known to those skilled in the art.

In certain embodiments, the TrkB agonizing antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade, 1989 J. Cline Pharmacol. 29:685). Exemplary targeting moieties include folate or biotin (see, e.g., U.S. Pat. No. 5,416,016 to Low et al.); mannosides (Umezawa et al., 1988 Biochem. Biophys. Res. Commun. 153:1038); antibodies (P. G. Bloeman et al., 1995 FEBS Lett. 357:140; M. Owais et al., 1995 Antimicrob. Agents Chernother. 39:180); surfactant protein A receptor (Briscoe et al., 1995 Am. J. Physiol. 1233:134); p120 (Schreier et al., 1994 J. Biol. Chem. 269:9090); see also K. Keinanen; M. L. Laukkanen, 1994 FEBS Lett. 346:123; J. J. Killion; I. J. Fidler, 1994 Immunomethods 4:273.

### Mouse Models

Mecp2 KO (Mecp2-knock out) males and Mecp2 HT (heterozygote) females are a very useful model system in which to study Rett Syndrome (RTT), as these organisms show similar symptoms to girls affected by RTT. Mecp2-KO males become symptomatic at 4 weeks, exhibiting any number of the following symptoms: growth decline; reduced brain growth and neuron size; tremors; motor impairment; hypoactivity (seizures); breathing irregularities; heightened anxiety; kyphosis; stereotypic forelimb motions; and hind limb clasping.

Mecp2 deficiencies in mice are known to be associated with low endogenous levels of BDNF and to disrupt the mice respiratory system, and specifically are related to progressive deficiency in norepinephrine and serotonin content leading to a misregulation of the medullary respiratory system. (Viemari et al., (2005) J Neuroscience; 25:11521). Said disruptions and respiratory difficulties are exhibited to a greater extent in Mecp2-KO mice. The central autonomic dysfunctions seen in these mice include progressively worsening breathing disturbances (erratic breathing pattern, variable cycle and frequent apneas) resulting in fatal respiratory arrest at ˜2 months of age; prolonged cardiac QT interval; and a drastic reduction in tyrosine hydroxylase, norepinephrine and serotonin content in the brainstem medulla, leading to an imbalance in the inhibitory system modulating the medullary respiratory network.

Mecp2 HT females show a similar but delayed phenotype (onset=3 months), which is attenuated with no rapid deterioration. They are capable of surviving for 9-12 months. The female Mecp2-HT, however, also present a respiratory phenotype characterized by a larger tidal and lung volume, respiratory depression, prolonged apnea following hyperventilation, and a greater response to hypoxia (Bissonnette and Knopp, (2006) Pediatric Research; 59:513).

The invention having been fully described, it is further illustrated by the following examples and claims, which are illustrative and are not meant to be further limiting. Those skilled in the art will recognize or be able to ascertain using no more than routine experimentation, numerous equivalents to the specific procedures described herein. Such equivalents are within the scope of the present invention and claims. The contents of all references, including issued patents and published patent applications, cited throughout this application are hereby incorporated by reference.

### Examples

### Example 1

### Administration of TrkB Agonist Antibody (C20) to Mecp2 Mice

An TrkB Agonist Antibody (C20) or saline was given intraperitoneally twice a week to Mecp2-KO and wild type males from 4 (early symptomatic) to 8 weeks (late symptomatic) of age at the dose of 3 mg/kg body weight and the animals were tested between 6 and 8 weeks. There were 9 to 14 mice per group tested, with four groups overall (wild type with saline, wild type with mAb administered, knockout with saline, and knockout with mAb administered). Mecp2 knockout mice were purchased from Jackson Labs (line B6.129P2©Mecp2^{tm1.1Bird}/J (#003890)).

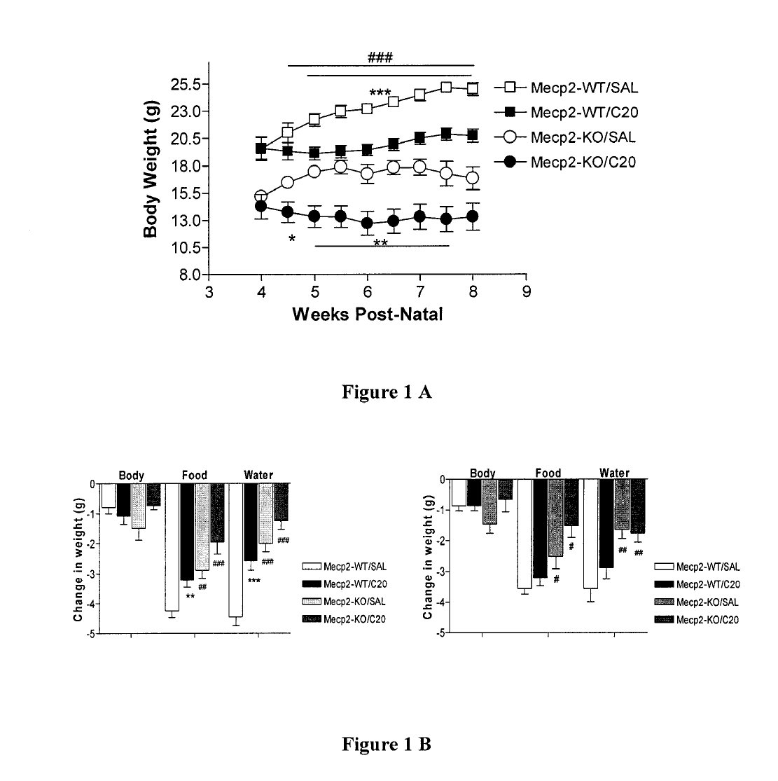

As seen in FIG. 1A, the mice showed a drop in body weight when TrkB agonist mAb was administered. The top line (with white square icons as data points) represents the Mecp2 wild type mice with saline administered. The second to top line (with black square icons as data points) represents the Mecp2 wild type mice with TrkB agonist antibodies administered. The second to bottom line (with white circle icons as data points) represents the Mecp2 knockout mice with saline administered. The bottom line (with black circle icons as data points) represents the Mecp2 knockout mice with TrkB agonist antibodies administered. As shown in FIG. 1A, the Mecp2 mice lost weight in both instances when the TrkB agonist antibodies were administered.

In the testing at both 6 (left in FIG. 1B) and 8 weeks (right in FIG. 1B), the mice to which the TrkB agonist mAb was administered also showed drops in food and water intake, when measured over a 24-hour period.

Furthermore, as seen in FIG. 1, the mice also showed a drop in body weight when TrkB agonist mAb was administered. The top line (with white square icons as data points) represents the Mecp2 wild type mice with saline administered. The second to top line (with black square icons as data points) represents the Mecp2 wild type mice with TrkB agonist antibodies administered. The second to bottom line (with white circle icons as data points) represents the Mecp2 knockout mice with saline administered. The bottom line (with black circle icons as data points) represents the Mecp2 knockout mice with TrkB agonist antibodies administered. As shown in FIG. 1, the Mecp2 mice lost weight in both instances when the TrkB agonist antibodies were administered.

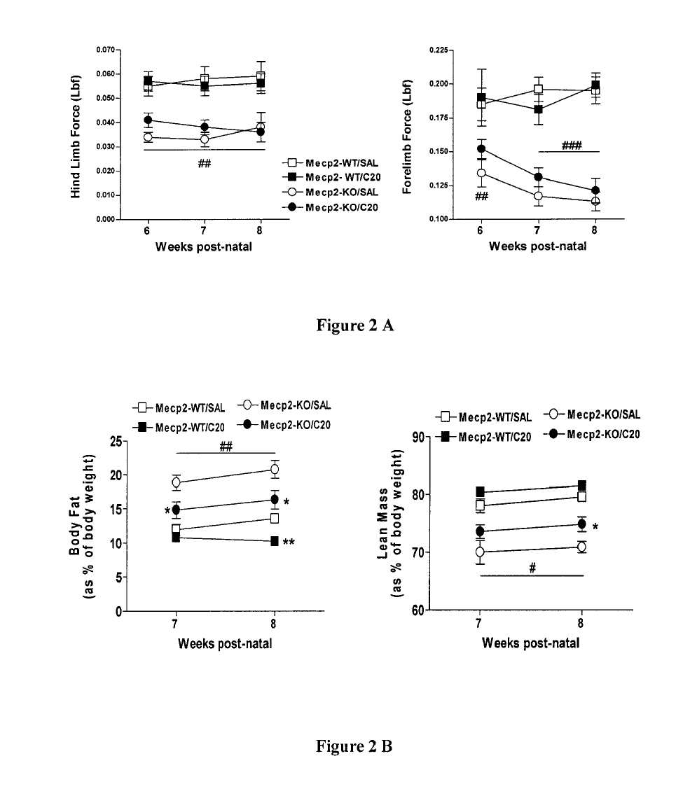

Furthermore, administration of the TrkB agonist mAb is able to improve the forelimb and hind limb grip strength of the treated-KO mice. This is demonstrated in FIG. 2A, where the square icons as data points represent the wild type (WT) mice treated with saline (SAL) or the TrkB agonist antibody C20; the circle icons as data points represent the knockout (KO) mice treated with saline (SAL) or the TrkB agonist antibody, C20. In addition, the mice to which the TrkB agonist antibody was given showed a decrease in body fat and an increase in lean mass content. This is demonstrated in FIG. 2B, where the square icons as data points represent the wild type (WT) mice treated with saline (SAL) or the TrkB agonist antibody C20; the circle icons as data points represent the knockout (KO) mice treated with saline (SAL) or the TrkB agonist antibody, C20.

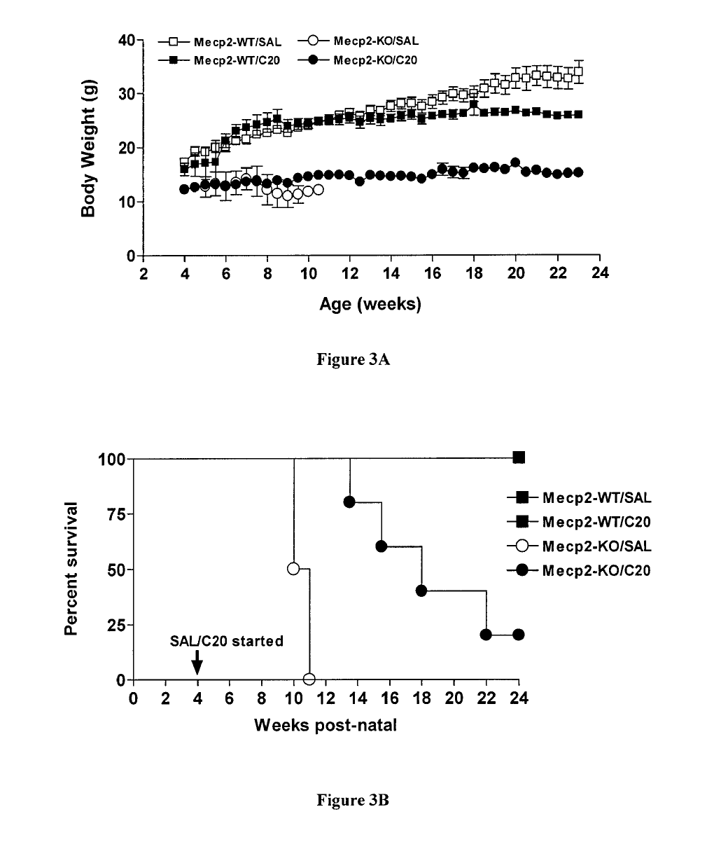

Administration of the TrkB agonist mAb is also able to increase longevity of Mecp2-KO mice. Mecp2-KO mice are known to expire roughly between 8 and 10 weeks, yet are capable of living longer when given TrkB agonist mAbs. This is demonstrated at least in FIG. 3A, in which the KO mice administered with saline (KO/SAL) die around 8-10 weeks of age, wherease the KO mice given the TrkB agonist mAb survive until 23 weeks of age (KO/C20); and in FIG. 3, in which the wild type mice are described as WT and the knockout mice are described as KO, the TrkB agonist antibody is described as C20. As shown in the FIG. 3, the TrkB agonist mAb-treated mice (KO/MAB) are able to survive to at least twice the age of the saline treated KO mice. The fatal breathing disturbances of Mecp2-KO mice are thought to be rescued via stimulation by TrkB agonist antibodies of TrkB expressed in the medullary respiratory network system; said system is negatively impacted in mouse and human when Mecp2 is absent and/or mutated.

Additionally, the TrkB agonist antibodies are thought to access the neurons of the medullary respiratory system and thereby restore normal levels of tyrosine hydroxylase (the rate-limiting enzyme for norepinephrine synthesis), norepinephrine and serotonin, thus preventing the respiratory deficits, and prolonging the lifespan, of the KO mice. These respiratory and related deficits have been studied and are related to progressive deficiency in norepinephrine and serotonin modulation of the medullary respiratory system (Viemari et al., (2005) J Neuroscience; 25:11521). Chronic treatment with the norepinephrine reuptake inhibitor, desipramine, can rescue this phenotype and significantly prolong the lifespan of Mecp2 KO mice (Roux et al. (2007) Eur. J. Neuroscience; 25:1915). Alternatively, the TrkB agonist antibodies are believed to bind to TrkB receptors located on neurons composing the carotid bodies and reestablish a disrupted transmission to higher functions in the brain (i.e., cortical or hypothalamic) that regulate respiratory patterns. Finally, the TrkB agonist antibodies are thought to act on TrkB receptors of the nodose cranial sensory ganglia and compensate for the decrease in BDNF reported in this structure that is critical for cardiorespiratory homeostasis (Ogier et al., (2007) J. Neuroscience; 27:10912).

TrkB agonist antibodies of the present methods can act in the same fashion, via the same or similar mechanisms (e.g., can act on reestablishing a normal level and balance of these neurotransmitters in the brainstem medulla). Radio-imaging with [3H]-labeled TrkB agonist antibodies can further confirm these findings, and can further elucidate possible mechanisms. As described elsewhere herein, TrkB agonist antibodies of the present methods can in some embodiments be combined with desipramine for additive efficacy.

### Example 2

### Administration of TrkB Agonist Antibody (C20) to Mecp2 Mice

For testing of apneas, whole body plethysmography can be employed, using a system designed by Buxco Research Systems. Conscious, unrestrained mice are placed into plethysmograph chambers (upright plexiglass cylinders 4 inches in diameter and 5 inches high). Airflow is maintained to ensure a constant exchange of fresh air into the chambers, and food, bedding, and water is provided. To accurately assess frequency of apneas, mice need to acclimate to the plethysmograph chambers. Once they have fully acclimated, airway response recordings are performed at various intervals without the need to remove animals from the chambers. The acclimation period and recording periods should not exceed two hours; therefore, the mice will not be in the chambers for more than two hours at a time.

Plethysmograph recordings are performed no more than twice a week in the same animal. Once plethysmograph recordings are completed, mice are returned to their home cage. If, while in the plethysmograph chamber, mice exhibit severely constrained breathing or obvious signs of anxiety, they can be removed from the chamber and returned to their home cage.

1. A method to treat, diagnose, prevent, or ameliorate a condition associated with respiratory disorders, comprising administering to a subject in need thereof an effective amount of a TrkB binding molecule.

2. The method of claim 1, wherein said TrkB binding molecule is a TrkB agonizing antibody.

3. The method of claim 2, wherein said antibody is a humanized antibody.

4. The method of claim 2, wherein said antibody does not bind to Tyrosine Kinase Recptor A or Tyrosine Kinase Receptor C.

5. The method of claim 2, wherein said antibody does not bind to the Ligand Binding Domain (LBD) of TrkB.

6. The method of claim 2, wherein said antibody does not compete with the binding of Brain Derived Neurotrophic Factor (BDNF) to TrkB.

7. The method of claim 2, wherein the antibody competes for binding to TrkB with a competitor antibody comprising a heavy chain variable region comprising SEQ ID NO:1 and a light chain variable region comprising SEQ ID NO:2.

8. The method of claim 2, wherein the antibody comprises a heavy chain variable region comprising SEQ ID NO:1 and a light chain variable region comprising SEQ ID NO:2.

9. The method of claim 2, wherein the antibody comprises a heavy chain variable region comprised of one or more of SEQ ID NO:5, SEQ ID NO:9, and SEQ ID NO:13, and a light chain variable region comprised of one or more of SEQ ID NO:6, SEQ ID NO:10, and SEQ ID NO:14.

10. The method of claim 2, wherein said antibody binds to the Ligand Binding Domain (LBD) of TrkB.

11. The method of claim 2, wherein said antibody competes with the binding of Brain Derived Neurotrophic Factor (BDNF) to TrkB.

12. The method of claim 2, wherein the antibody competes for binding to TrkB with a competitor antibody comprising a heavy chain variable region comprising SEQ ID NO:3 and a light chain variable region comprising SEQ ID NO:4.

13. The method of claim 2, wherein the antibody comprises a heavy chain variable region comprising SEQ ID NO:3 and a light chain variable region comprising SEQ ID NO:4.

14. The method of claim 2, wherein the antibody comprises a heavy chain variable region comprised of one or more of SEQ ID NO:7, SEQ ID NO:11, and SEQ ID NO:15, and a light chain variable region comprised of one or more of SEQ ID NO:8, SEQ ID NO:12, and SEQ ID NO:14.

15. A method to treat, diagnose, prevent or ameliorate symptoms of respiratory distress, comprising administering to a subject in need thereof an effective amount of a TrkB binding molecule.

16. The method of claim 15, wherein said TrkB binding molecule is a TrkB agonizing antibody.

17. The method of claim 16, wherein said antibody is a humanized antibody.

18. The method of claim 16, wherein said antibody does not bind to Tyrosine Kinase Recptor A or Tyrosine Kinase Receptor C.

19. The method of claim 16, wherein said antibody does not bind to the Ligand Binding Domain (LBD) of TrkB.

20. The method of claim 16,'wherein said antibody does not compete with the binding of Brain Derived Neurotrophic Factor (BDNF) to TrkB.

21. The method of claim 16, wherein the antibody competes for binding to TrkB with a competitor antibody comprising a heavy chain variable region comprising SEQ ID NO:1 and a light chain variable region comprising SEQ ID NO:2.

22. The method of claim 16, wherein the antibody comprises a heavy chain variable region comprising SEQ ID NO:1 and a light chain variable region comprising SEQ ID NO:2.

23. The method of claim 16, wherein the antibody comprises a heavy chain variable region comprised of one or more of SEQ ID NO:5, SEQ ID NO:9, and SEQ ID NO:13, and a light chain variable region comprised of one or more of SEQ ID NO:6, SEQ ID NO:10, and SEQ ID NO:14.

24. The method of claim 16, wherein said antibody binds to the Ligand Binding Domain (LBD) of TrkB.

25. The method of claim 16, wherein said antibody competes with the binding of Brain Derived Neurotrophic Factor (BDNF) to TrkB.

26. The method of claim 16, wherein the antibody competes for binding to TrkB with a competitor antibody comprising a heavy chain variable region comprising SEQ ID NO:3 and a light chain variable region comprising SEQ ID NO:4.

27. The method of claim 16, wherein the antibody comprises a heavy chain variable region comprising SEQ ID NO:3 and a light chain variable region comprising SEQ ID NO:4.

28. The method of claim 16, wherein the antibody comprises a heavy chain variable region comprised of one or more of SEQ ID NO:7, SEQ ID NO:11, and SEQ ID NO:15, and a light chain variable region comprised of one or more of SEQ ID NO:8, SEQ ID NO:12, and SEQ ID NO:14.

29. A method to treat, diagnose, prevent, or ameliorate a condition associated with Rett Syndrome (RTT), comprising administering to a subject in need thereof an effective amount of a TrkB binding molecule.

30. The method of claim 29, wherein said TrkB binding molecule is a TrkB agonizing antibody.

31. The method of claim 30, wherein said antibody is a humanized antibody.

32. The method of claim 30, wherein said antibody does not bind to Tyrosine Kinase Recptor A or Tyrosine Kinase Receptor C.

33. The method of claim 30, wherein said antibody does not bind to the Ligand Binding Domain (LBD) of TrkB.

34. The method of claim 30, wherein said antibody does not compete with the binding of Brain Derived Neurotrophic Factor (BDNF) to TrkB.

35. The method of claim 30, wherein the antibody competes for binding to TrkB with a competitor antibody comprising a heavy chain variable region comprising SEQ ID NO:1 and a light chain variable region comprising SEQ ID NO:2.

36. The method of claim 30, wherein the antibody comprises a heavy chain variable region comprising SEQ ID NO:1 and a light chain variable region comprising SEQ ID NO:2.

37. The method of claim 30, wherein the antibody comprises a heavy chain variable region comprised of one or more of SEQ ID NO:5, SEQ ID NO:9, and SEQ ID NO:13, and a light chain variable region comprised of one or more of SEQ ID NO:6, SEQ ID NO:10, and SEQ ID NO:14.

38. The method of claim 30, wherein said antibody binds to the Ligand Binding Domain (LBD) of TrkB.

39. The method of claim 30, wherein said antibody competes with the binding of Brain Derived Neurotrophic Factor (BDNF) to TrkB.

40. The method of claim 30, wherein the antibody competes for binding to TrkB with a competitor antibody comprising a heavy chain variable region comprising SEQ ID NO:3 and a light chain variable region comprising SEQ ID NO:4.

41. The method of claim 30, wherein the antibody comprises a heavy chain variable region comprising SEQ ID NO:3 and a light chain variable region comprising SEQ ID NO:4.

42. The method of claim 30, wherein the antibody comprises a heavy chain variable region comprised of one or more of SEQ ID NO:7, SEQ ID NO:11, and SEQ ID NO:15, and a light chain variable region comprised of one or more of SEQ ID NO:8, SEQ ID NO:12, and SEQ ID NO:14.

43. (canceled)

44. (canceled)

45. (canceled)
